(19) United States
(12) Patent Application Publication
(10) Pub. No.: US 2015/0031571 A1
(43) Pub. Date: Jan. 29, 2015

(54) CHEMILUMINESCENT NANOPARTICLES AND USES THEREOF

(71) Applicants: UNIVERSITY OF KANSAS, LAWRENCE, KS (US); KANSAS STATE UNIVERSITY RESEARCH FOUNDATION, MANHATTAN, KS (US)

(72) Inventors: Judy Wu, Lawrence, KS (US); Mark Richter, Lecompton, KS (US); Lateef U. Syed, Hillsboro, OR (US); Jun Li, Manhattan, KS (US); Scott Hefty, Lawrence, KS (US)

(73) Assignees: UNIVERSITY OF KANSAS, LAWRENCE, KS (US); KANSAS STATE UNIVERSITY RESEARCH FOUNDATION, MANHATTAN, KS (US)

(21) Appl. No.: 14/377,019

(22) PCT Filed: Feb. 7, 2013
(86) PCT No.: PCT/US2013/025120

§ 371 Date: Aug. 6, 2014

Related U.S. Application Data
(60) Provisional application No. 61/595,958, filed on Feb. 7, 2012.

(51) Int. Cl. G01N 33/58; C12Q 1/68; G01N 21/76; C12Q 1/70

(52) U.S. Cl. CPC G01N 33/582; C12Q 1/707; C12Q 1/689; G01N 21/76
USPC 506/9; 422/52; 435/287.2; 506/16; 435/34; 435/6.11; 435/5

(57) ABSTRACT

Gold nanoparticles having luminol covalently linked thereto and optionally functionalized with an oligonucleotide and bacterial or viral detection assays. In one aspect, the detection system for detecting an analyte in a sample comprises a light-shielding container having a fiberoptic cable for transmitting light generated within the light-shielding container to a photodetector; a plurality of functionalized nanoparticles deposited in solid form on or within a support, such that the support is located within the light-shielding container; wherein the functionalized nanoparticles comprise nanoparticles covalently attached to one or more chemiluminescent moieties; and a reagent system which causes the chemiluminescent moieties to produce light in the presence of the reagent system and the analyte in the sample.

### CROSS-REFERENCE TO RELATED APPLICATIONS

This application is based on and claims priority to U.S. Provisional Application Ser. No. 61/595,958, filed on Feb. 7, 2012, which is hereby incorporated herein by reference.

### BACKGROUND OF THE INVENTION

1. Field of the Invention

The present invention is directed to compositions of matter for use in devices for the chemiluminescent-based detection of analytes.

2. Description of Related Art

Chemiluminescence is a process in which visible light is emitted as a result of chemical reactions. It has been widely utilized for forensic investigations by spraying luminol (5-amino-2,3-dihydro-1,4-phthalazine-dione) to identify dried bloodstains. Recently, luminol was attached to gold nanoparticles using 3-mercaptopropionic acid and then functionalized with antibodies (luminol-AuNP-Ab) and stored in solution at 4° C. for use in a sandwich immunoassay for the detection of carcinoembryonic antigen in serum involving antibody immobilized magnetic beads (MBs-Ab) as described in Yang et al., Luminol/antibody labeled gold nanoparticles for chemiluminescence immunoassay of carcinoembryonic antigen, Anal Chim Acta 666 (1-2) 91-96 (2010). Such an assay requires the luminol-AuNP-Ab solution to be refrigerated, requires expensive equipment to perform, and requires incubation of times of several hours to permit the immunocomplex to form. The poor stability, short shelf life, and lack of specificity to particular targets (microbes or pathogens) of antibodies limit this method for broad applications. Thus, there remains a need for improved chemiluminescent-based detection systems.

### BRIEF SUMMARY OF THE INVENTION

The present invention is directed to novel chemiluminescent nanoparticles and their use in chemiluminescent detection systems. In one aspect, the detection system for detecting an analyte in a sample comprises a light-shielding container having a fiberoptic cable for transmitting light generated within the light-shielding container to a photodetector; a plurality of functionalized nanoparticles deposited in solid form on or within a support, such that the support is located within the light-shielding container; wherein the functionalized nanoparticles comprise nanoparticles covalently attached to one or more chemiluminescent moieties; and a reagent system which causes the chemiluminescent moieties to produce light in the presence of the reagent system and the analyte in the sample.

In another aspect, the detection system is designed to detecting a bacterium or virus in a sample and comprises a light-shielding container having a fiberoptic cable for transmitting light generated within the light-shielding container to a photodetector; a support located within the light-shielding container, the support having a sample application region, a test region, and a control region; a plurality of first functionalized nanoparticles deposited in solid form on or within the sample application region of the support, wherein the first functionalized nanoparticles comprise nanoparticles covalently attached to a chemiluminescent moiety and a first oligonucleotide probe capable of selectively hybridizing to the bacterium or virus nucleic acids; a plurality of second particles functionalized with a second oligonucleotide probe capable of selectively hybridizing to the bacterium or virus nucleic acids, the second particles immobilized on or within the test region of the support; a plurality of third particles functionalized with a third oligonucleotide probe capable of selectively hybridizing to the first oligonucleotide probe, the third particles immobilized on or within the control region of the support; and a reagent system which causes the chemiluminescent moiety to produce light in the presence of the reagent system and the first functionalized nanoparticles.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a schematic illustration of nanoparticles functionalized with a chemiluminescent molecule (“CL-NP”) (panel a) and with an additional probe functionalization (“Probe-CL-NP”) (panel b).

FIG. 2A is a schematic illustration of two exemplary embodiments of the nanoparticle-supported chemiluminescence as low-cost portable systems. Panel a illustrates an exemplary multiwell format for analyte detection (such as blood detection) using the CL-NPs shown in FIG. 1, panel a. Panel b illustrates a test strip format for a biosensor to detect nucleic acids or other analytes using the Probe-CL-NPs shown in FIG. 1, panel b.

FIG. 2B shows another embodiment of a multiwell system. A workstation enclosing a highly sensitive charge coupled device (CCD) as photon detector, optics (lens, filters, etc.) to collect photons, and sample stages in a larger black box. An example is the IVIS Lumina II system by Caliper Inc.

FIG. 2C shows the details of an exemplary test strip (e.g., for detecting a bacterium or viral analyte) for use in the system illustrated in panel b of FIG. 2A.

FIG. 3 summarizes the scheme for the modification of gold nanoparticles (“GNPs”) with luminol through a linker 11-mercaptoundecanoic acid (“MUA”) to form conjugated nanoparticles I (“GNP-MUA-LUM”).

FIG. 4 shows two schemes for implementing chemiluminescent-functionalized nanoparticles (“I” or “GNP-MUA-LUM”) for biosensing. In Scheme I, gold nanoparticles with luminol are directly used to detect analytes (such as red blood cells) which can catalyze the chemiluminescence. In Scheme II, light from luminol and probe co-functionalized gold nanoparticles are bound to an immobilized second probe on a test strip through a specific biomarker (e.g., nucleic acids) containing nonoverlapped binding sites (or epitopes), and the light is measured by supplying the sample with proper reagents for chemiluminescence.

FIG. 5 shows the UV-visible (panel a) and FT-IR spectra (panel b) of citrate-stabilized GNPs, MUA-modified GNPs after replacing citrate, and LUM attached GNPs. The small peak at 347 nm in the GNP-MUA-LUM curve of panel a corresponds to an absorption peak of LUM. The GNP-MUA and GNP-MUA-LUM curves in panel a were translated upward by 0.2 and 0.5 units, respectively, along the y-axis for better comparison. Also, the GNP-MUA and GNP-MUA-LUM curves in panel b were translated upward by 20 and 50 units, respectively, along the y-axis for clearer visualization.

FIG. 6 shows the TEM images of citrate stabilized GNPs (panel a), MUA-modified GNPs (panel b), and luminol-modified GNPs (panel c). The scale bars for panels a-c is 20, 50, and 50 nm respectively. Panels d-f show size distributions of GNPs at various stages of modification. The average diameter of citrate stabilized, MUA, and luminol-modified GNPs is about 9.8, about 8.8, and about 9.2 nm, respectively.

FIG. 7 shows the chemiluminescent signal recorded using IVIS Lumina II. Panels a and b are snapshot images using pseudocolor to represent the chemiluminescent intensities from two designated PDMS wells on a glass slide, which are loaded with about 1×10¹⁰ and about 1×10³ GNP-MUA-LUM, respectively. Panels c and d show plots of the integrated chemiluminescent signal (filled circles) from the wells containing about 1.0×10¹⁰ and about 1.0×10³ GNP-MUA-LUM over background signal (filled squares) obtained in control experiments without Fe(CN)₆³⁻ ions.

FIG. 8 is a comparison of kinetic plots of the chemiluminescent signal of 1.0×10¹⁰ luminol-functionalized gold nanoparticles in a PDMS well (filled triangles), the same amount of luminol molecules dispersed in the solution (filled squares), and blank control sample (filled circles). Each 10 nm diameter gold nanoparticle is estimated to be attached with about 1.4×10³ luminol molecules by assuming the formation of a close-packed monolayer with the same density as that on the flat gold surface. The amount of luminol on 1.0×10¹⁰ luminol-functionalized gold nanoparticles is equivalent to 4.04 μL of 23 μM luminol solution used for comparison. The absorption of gold nanoparticles was not corrected.

FIG. 9 is a calibration curve obtained by plotting a log-log plot of background subtracted peak chemiluminescence (ΔIₘₐₓ) from 1 mM Fe(CN)₆³⁻ solution with varying number of luminol-modified GNPs. The solid line is the best fit line, which shows nice linearity.

FIG. 10 (panel a) shows the UV-visible absorption spectra measured with varying number of GNP-MUA-LUM in 350 μL solution in a microcuvette with an optical pathlength of 10.0 mm. Panel b shows the enlarged view to show the absorption spectra of highly diluted GNP-MUA-LUM solutions.

FIG. 11 shows the background subtracted peak absorption (Aₚₑₐₖ) at about 520 nm derived from the UV-visible spectra. Panel a plots the value of Aₚₑₐₖ vs. the logarithm of the number of luminol-labeled GNPs. Panel b is the linear plot of Aₚₑₐₖ vs. number of luminol-labeled GNPs obtained with the sample containing about 1.0×10¹⁰, 1.0×10⁹, and 1.0×10⁸ GNP-MUA-LUM. The solid line in panel b is the best fit line, which fits nicely with a linear equation. This indicates that the UV-visible signal linearly decreases till 10⁸ GNPs. At higher dilution (i.e., the number of GNP-MUA-LUM < about 10⁸) the samples did not show reliable UV-visible signal (as evident from FIG. 10). The detection limit by UV-visible absorption is clearly about 10⁷ to 10⁸ GNPs/well.

FIG. 12 shows the kinetic plots of the chemiluminescent signal (filled circles) of lysed (panels a and b) and unlysed (panels c and d) blood samples. Panels a and c were measured at stock concentration and panels b and d were measured after 10⁸ fold dilution. The black squares represent the background from control experiments without adding any blood sample.

FIG. 13 shows the calibration curves of the lysed and unlysed blood samples on the Log-Log scale of ΔIₘₐₓ (the background subtracted peak chemiluminescent intensity in kinetic measurements) vs. the dilution factor. The solid lines are linear fitting to two regions.

FIG. 14 shows the comparison of chemiluminescence signal of luminol molecules (LUM) in bulk aqueous solutions and equivalent amount of luminol molecules covalently attached to 10-nm diameter gold nanoparticles (GNPs).

FIG. 15 shows the calibration curve of chemiluminescence signal vs. Fe³⁺ catalyst concentration in bulk luminol solutions.

### DETAILED DESCRIPTION OF PREFERRED EMBODIMENT

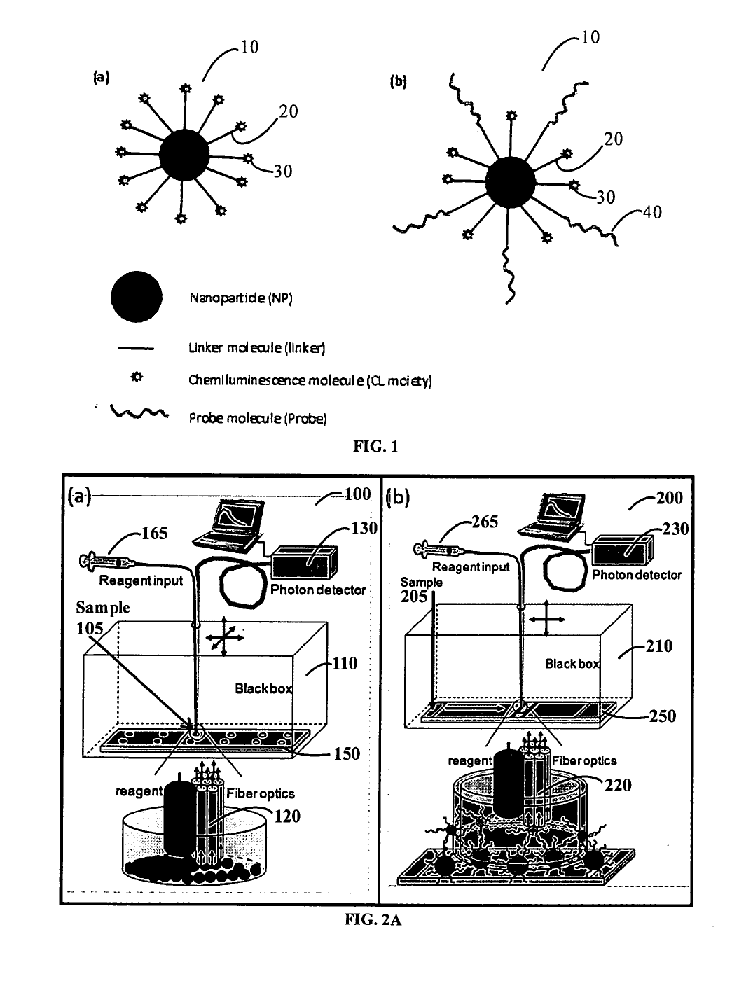

The present invention is directed to chemiluminescent compositions of matter comprising nanoparticles 10 covalently attached via one or more linkers 20 to one or more chemiluminescent moieties 30 that are optionally further functionalized with one or more probe moieties 40. The chemiluminescent compositions of matter of the present invention are generally shown in FIG. 1.

The nanoparticles of the present invention are preferably less than about 1 micron in size, for example, about 1000, 900, 800, 700, 600, 500, 400, 300, 200, or 100 nm. In some embodiments, the average or mean diameter of the nanoparticles is between about 2 to about 100 nm, and most preferably between about 2 to 50 nm. In some embodiments, the average or mean diameter of the nanoparticles is about 2, 3, 4, 5, 6, 7, 8, 9, 10, 15, 20, 25, 30, 35, 40, 45, 50, 60, 70, 80, 90, or 100 nm (or some range therebetween).

The nanoparticles may have various morphologies or structures. Non-limiting examples of suitable regular shapes of the metal nanoparticles include spheres, oblate spheres, prolate spheroids, ellipsoids, rods, cylinders, cones, disks, cubes, and rectangles. Preferably, the nanoparticles are generally spherical in shape.

The nanoparticles may be comprised of various materials used in conventional diagnostic assays. Non-limiting examples include Al₂O₃, TiO₂, ZrO₂, Y₂O₃, SiO₂, ferric oxide, ferrous oxide, a rare earth metal oxide, a transitional metal oxide, mixtures thereof, and alloys thereof. Additional non-limiting examples of metals include aluminum, gold, silver, stainless steel, iron, titanium, cobalt, nickel, and alloys thereof. In certain embodiments, nanoparticle may be comprised of biodegradable polymers, non-biodegradable water-soluble polymers, non-biodegradable non-water soluble polymers, and biopolymers. Non-limiting examples include such materials as poly(styrene), poly(urethane), poly(lactic acid), poly(glycolic acid), poly(ester), poly(alpha-hydroxy acid), poly(epsilon-caprolactone), poly(dioxanone), poly(orthoester), poly(ether-ester), poly(lactone), poly(carbonate), poly(phosphazene), poly(phosphonate), poly(ether), poly(anhydride), mixtures thereof and copolymers thereof. In one aspect, the nanoparticles are preferably comprised of metals, and most preferably, the nanoparticles used in the compositions of the present invention are comprised of gold. It is believed that the chemiluminescent signal is enhanced due to (a) charge transfer at the gold nanoparticles, and (b) aggregation of the gold nanoparticles.

The linker is preferably a linear molecule with functional groups at the two ends, one of which can form covalent bond with the nanoparticles and the other one is preferably a carboxylic group. The example linker to be used on gold nanoparticles is preferably derived from carboxylic acid having a terminal thiol group. Exemplary carboxylic acids include the C₅ to C₂₀ carboxylic acids (e.g., C₅, C₆, C₇, C₈, C₉, C₁₀, C₁₁, C₁₂, C₁₃, C₁₄, C₁₅, C₁₆, C₁₇, C₁₈, C₁₉, or C₂₀), such as mercaptoundecanoic acid (“MUA”). A carboxy activating agent is used for the coupling of primary amines in the chemiluminescent material to yield amide bonds. Preferably, diimides and amine-reactive N-hydro-succinimide esters, such as 1-ethyl-3-(3-dimethylaminopropyl) carbodiimide hydrochloride (“EDC”) and N-hydrosuccinimide (“NHS”), are used for this coupling step. The linker may be used to attach the chemiluminescent moiety to the nanoparticle and/or to attach the probe moiety to the nanoparticle.

Suitable chemiluminescent agents include amine-reactive luminol derivatives, microperoxidasies, acridinium esters, peroxidases, and derivatives thereof. The chemiluminescent moiety is preferably a diacylhydrazides. Exemplary chemiluminescent moieties include, but are not limited to, luminol, N-(4-aminobutyl)-N-ethylisoluminol, 4-aminophthalhydrazide monohydrate, bis(2-carbopentyloxy-3,5,6-trichlorophenyl) oxalate, 9,10-bis(phenylethynyl)anthracene, 5,12-bis(phenylethynyl)naphthacene, 2-chloro-9,10-bis(phenylethynyl)anthracene, 1,8-dichloro-9,10-bis(phenylethynyl)anthracene, Lucifer Yellow CH dipotassium salt, Lucifer yellow VS dilithium salt, 85% (dye content), 2,4,5-Triphenylimidazole, 9,10-Diphenylanthracene, Rubrene, or Tetrakis(dimethylamino)ethylene.

The chemiluminescent nanoparticles may also optionally include a probe moiety having a recognition portion that can recognize and bind to the target of interest (analyte). In the present invention, the nanoparticles are preferably functionalized with an oligonucleotide probe that is complementary to and capable of selectively hybridizing to a target polynucleotide. The term “hybridization” as used herein refers to the process in which two single-stranded polynucleotides bind non-covalently to form a stable double-stranded polynucleotide; triple-stranded hybridization is also theoretically possible.

The oligonucleotide probe is preferably capable of selectively hybridizing to a segment of the nucleic acid contents (the analyte) in a target bacterium or virus. The target virus may be single or double stranded or DNA-based or RNA-based. In one aspect, the target virus is selected from the group consisting of Parvoviridae, Papovaviridae, Adenoviridae, Hepadnaviridae, Herpesviridae, Iridoviridae, and Poxyiridae. For example, it is intended that the present invention encompass methods for the detection of any DNA-containing virus, including, but not limited to Hepatitis B, parvoviruses, dependoviruses, papillomaviruses, polyomaviruses, mastadenoviruses, aviadenoviruses, hepadnaviruses, simplexviruses (such as herpes simplex virus 1 and 2), varicelloviruses, cytomegaloviruses, muromegaloviruses, lymphocryptoviruses, thetalymphocryptoviruses, rhadinoviruses, iridoviruses, ranaviruses, pisciniviruses, orthopoxviruses, parapoxviruses, avipoxviruses, capripoxviruses, leporipoxviruses, suipoxviruses, yatapoxviruses, and mulluscipoxvirus). Thus, it is not intended that the present invention be limited to any DNA virus family. In further embodiments, the target virus is selected from the group consisting of Picornaviridae, Caliciviridae, Reoviridae, Togaviridae, Flaviviridae, Orthomyxoviridae, Paramyxoviridae, Arenaviridae, Rhabdoviridae, Coronaviridae, Bunyaviridae, and Retroviridae. For example, it is intended that the present invention encompass methods for the detection of RNA-containing virus, including, but not limited to enteroviruses (e.g., polioviruses, Coxsackieviruses, echoviruses, enteroviruses, hepatitis A virus, encephalomyocarditis virus, mengovirus, rhinoviruses, and aphthoviruses), caliciviruses, reoviruses, orbiviruses, rotaviruses, Birnaviruses, alphaviruses, rubiviruses, pestiviruses, flaviviruses (e.g., hepatitis C virus, yellow fever viruses, dengue, Japanese, Murray Valley, and St. Louis encephalitis viruses, West Nile fever virus, Kyanasur Forest disease virus, Omsk hemorrhagic fever virus, European and Far Eastern tick-borne encephalitis viruses, and louping ill virus), influenza viruses (e.g., types A, B, and C), paramyxoviruses, morbilliviruses, pneumoviruses, veisculoviruses, lyssaviruses, filoviruses, coronaviruses, bunyaviruses, phleboviruses, nairoviruses, uukuviruses, hantaviruses, sarcoma and leukemia viruses, oncoviruses, HTLV, spumaviruses, lentiviruses, arenaviruses, and human immunodeficiency virus (HIV). Thus, it is not intended that the present invention be limited to any RNA virus family.

The target bacterium may be any suitable bacterial species (spp.), for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, Enterococcus faecum), Escherichia spp. (e.g., Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Pseudomonas spp. (e.g., Pseudomonas aeruginosa), Rickettsia spp. (e.g., Rickettsia rickettsii), Salmonella spp. (e.g., Salmonella enterica, Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (e.g., Yersinia pestis). Other bacterial species not listed above can also be detected as would be understood by one of skill in the art.

The oligonucleotide probe is typically comprised of 10 to 100 deoxyribonucleotides or ribonucleotides, preferably about 20 to 50 nucleotides (e.g., 20, 22, 24, 26, 28, 30, 32, 34, 36, 38, 40, 42, 44, 46, 48, or 50 nt in length), and most preferably about 20 to 30 nucleotides.

In a preferred aspect, the oligonucleotide probe is capable of selectively hybridizing the RNAs of the Hepatitis C virus (“HCV”) or the reverse-transcripted DNAs. HCV is a member of the Flaviviridae family. More specifically, HCV has about 9.5 kb sized (+)-RNA (single stranded positive-sense RNA) genome inside its membrane. The RNA genome consists of an untranslational region (“UTR”) at 5′ and 3′ ends and a long open reading frame (“ORF”). This ORF is expressed as a polyprotein including 3,010 to 3,040 amino acids by host cell enzymes and divided into 3 structural proteins and 6 nonstructural proteins by the host cell and its own protease. Also, there is a uniformly conserved region in the 5′ and 3′ end of the genome, respectively. This region is believed to play an important role for protein formation and RNA replication of the virus. The long ORF is expressed as a polyprotein, and through co-translational or post-translational processing, it is processed into structural proteins, i.e., core antigen protein (core) and surface antigen protein (E1, E2), and nonstructural proteins, NS2 (protease), NS3 (serine protease, helicase), NS4A (serine protease cofactor), NS4B (protease cofactor, involved in resistance), NS5A, and NS5B (RNA dependent RNA polymerase, RdRp), each contributing to replication of virus. The structural proteins are divided into core, E1 and E2 by signal peptidase of the host cell. Meanwhile, the nonstructural proteins are processed by serine protease (“NS3”) and cofactor (“NS2,” “NS4A,” and “NS4B”) of the virus. The core antigen protein together with surface antigen protein of the structural protein compose a capsid of the virus, and the nonstructural proteins like NS3 and NS5B play an important part of the RNA replication of the virus (see Bartenschager, Molecular targets in inhibition of hepatitis C virus replication, Antivir. Chem. Chemother. 8 281-301 (1997)).

Similar to other Flaviviruses, the 5′ and 3′ ends of the virus RNA has a uniformly conserved untranslational region. Generally, this region is known to play a very important role in replication of the virus. The 5′ end has 5′-UTR composed of 341 nucleotides, and this part has the structure of 4 stem and loop (I, II, III, and IV). Actually, this part functions as an internal ribosome entry site (“IRES”) necessary for translation processing to express protein. Particularly, the stem III, which has the biggest and most stable structure and has a conserved sequence, has been reported to play the most essential part for ribosome binding. In addition, it is known that proteins of the virus are expressed by initiating translation processing from AUG that exists in the single RNA of the stem IV (see Stanley et al., Internal ribosome entry sites within the RNA genomes of hepatitis C virus and other Flaviviruses, seminars in Virology 8 274-288 (1997)). Moreover, the 3′ end has 3′-UTR composed of 318 nucleotides. This part is known to play a very important role in initiation step of binding of NS5B, an essential enzyme of RNA replication. The 3′-UTR, according to the sequence and tertiary structure, is composed of three different parts: -X-tail-5′ starting from the 5′ end to 98th nucleotide (98 nt), -poly(U)- having UTP consecutively, and the rest of 3′-UTR-. More specifically, X-tail-5′ part consists of 98 nucleotides having a very conserved sequence, and has three stem and loop structures, thereby forming a very stable tertiary structure. Probably, this is why X-tail-5′ part is considered very essential of NS5B binding. Also, it is reported that -poly(U)- part induces a pyrimidine track, thereby facilitating RNA polymerase effect. Lastly, the rest of the 3′-UTR has the tertiary structure of loop and plays an important role in NS5B binding. However, its structure is somewhat unstable. Overall, the 3′ end region of HCV RNA is known to have an essential structure in NS5B binding when the RNA replication starts (see Yamada et al., Genetic organization and diversity of the hepatitis C virus genome, Virology 223 255-281 (1996)). Oligonucleotide probes complementary to these regions are most preferred. Most preferred oligonucleotide probes are complementary to the X-tail and are set forth below:

| | |
|---|---|
| SEQ ID NO 1: | GTGGCCCCATCTTAGCCCTAGT |
| SEQ ID NO 2: | ACGGCTAGCTGTGAAAGGTCCGTGA |

It will be appreciated that the sequence of the oligonucleotide probe will be a function of the target virus or bacterium. Four exemplary sequences for the influenza hemagglutinin (HA) virus are as follows:

SEQ ID NO 3:
5′GTCTCCCTGGGGGCAATCAGTTTCTGGATGTGCTC3′
SEQ ID NO 4:
5′CAAATGCAGACACATTATGTATAGGTTATCATGC3′
SEQ ID NO 5:
5′ACAGTACTAGAAAAGAATGTAACAGTAACACACTCTGTTAA3′
SEQ ID NO 6:
5′AGAGAGCAATTGAGCTCAGTGTCATCATT3′

Likewise, three exemplary sequences for influenza neuraminidase (NA) are as follows:

SEQ ID NO 7:
5′CACTATGAGGAATGCTCCTGTTATCCTGAT3′
SEQ ID NO 8:
5′TCTAATGGAGCAAATGGAGTAAAAGGATT3′
SEQ ID NO 9:
5′GGCAATGGTGTTTGGATAGGGAGAACTAAAAG3′

Exemplary sequences for Chlamydia trachomatis DNA sequences are as follows:

SEQ ID NO 10:
TCGCATGCTCAATAGTGCGACTTGTGCTGCTGGCGGCATAGGA
TTGTTAACACCAGTGGTATGC (64 mer)
SEQ ID NO 11:
ATGAACACACTCAGTTTTAGAAACGCTTTTG (31 mer)
SEQ ID NO 12:
GGCAGTTGCTGTGGCCACTATATTGGCC (28 mer)
SEQ ID NO 13:
TAGCGGCATCTTTATTCTTCGGGGTAGG (28 mer)
SEQ ID NO 14:
TTGGAGGAGTGCTGACTACAGAAGCTGTGA (30 mer)
SEQ ID NO 15:
CATCGATCACAAACTTTGATGTGGAACAACTTATGCTGTAAAAC
(44 mer)
SEQ ID NO 16:
GCAGAGGTTGAGCAGAAAATCTCGACAGCTAGTGCAAATGCC
AAAAGCAATGATAAG (57 mer)

The probes may be of any length that would selectively hybridize to the target bacterium or virus, and for example may be, for example, about 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, or about 500 nucleotides in length. Probes may also include additional sequence at their 5′ and/or 3′ ends so that they extent beyond the target sequence with which they hybridize. Variant nucleotide sequences may also be used, such as those having about 75%, 80%, 85%, desirably about 90% to 95% or more, and more suitably about 98% or more sequence identity to that particular nucleotide sequence as determined by sequence alignment programs known in the art. The term “sequence identity” means that two polynucleotide sequences are identical (i.e., on a nucleotide-by-nucleotide basis) over the window of comparison. The term percentage of sequence identity means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Thus, in one aspect, the oligonucleotide probe comprises a nucleotide sequence that shares at least 75, 80, 85, 90, 95, 98, or 100% sequence identity with the sequence of any one of SEQ ID NO: 1 to 9.

The term “selectively hybridize” means to detectably and specifically bind. The probes selectively hybridize to nucleic acid strands under hybridization conditions that minimize appreciable amounts of detectable binding to nonspecific nucleic acids. High stringency conditions can be used to achieve selective hybridization conditions as known in the art and discussed herein.

In a preferred embodiment, the oligonucleotide probe is attached to the nanoparticle via a linker. The linker may be the same or different from the linker used to attach the chemiluminescent moiety to the nanoparticle. The linker for gold nanoparticles is preferably derived from carboxylic acid having a terminal thiol group. Exemplary carboxylic acids include the C₅ to C₁₈ carboxylic acids, such as MUA. A carboxy activating agent is for the coupling of primary amines in the chemiluminescent material to yield amide bonds. Preferably, diimides and amine-reactive N-hydro-succinimide esters, such as EDC and NHS are used for this coupling step. The linker may be used to attach the chemiluminescent agent to the nanoparticle and/or to attach the probe moiety to the nanoparticle.

In another aspect, the nanoparticles may be functionalized with an oligonucleotide probe as is generally described in Mirkin et al., U.S. Application No. 2009/0325812, which is incorporated by reference in its entirety. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex. pages 109-121 (1995). See also, Mucic et al., Synthesis and characterization of DNA with ferrocenyl groups attached to their 5′-termini: electrochemical characterization of a redox-active nucleotide monolayer, Chem. Commun. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor, and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g., Burwell, Chemical Technology, 4, 370-377 (1974) and Matteucci et al., Synthesis of deoxyoligonucleotides on a polymer support, J. Am. Chem. Soc. 103 3185-3191 (1981) for binding of oligonucleotides to silica and glass surfaces, and Grabar et al., Preparation and characterization of Au colloid monolayers, Anal. Chem. 67 735-743 (1995) for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc. 109, 2358 (1987) (disulfides on gold); Allara and Nuzzo, Langmuir 1, 45 (1985) (carboxylic acids on aluminum); Allara and Tompkins, J. Colloid Interface Sci. 49 410-421 (1974) (carboxylic acids on copper); Iler, The Chemistry Of Silica, Chapter 6 (Wiley 1979) (carboxylic acids on silica); Timmons and Zisman, J. Phys. Chem. 69 984-990 (1965) (carboxylic acids on platinum); Soriaga and Hubbard, J. Am. Chem. Soc. 104, 3937 (1982) (aromatic ring compounds on platinum); Hubbard, Acc. Chem. Res. 13, 177 (1980) (sulfolanes, sulfoxides and other functionalized solvents on platinum); Hickman et al., J. Am. Chem. Soc. 111, 7271 (1989) (isonitriles on platinum); Maoz and Sagiv, Langmuir 3, 1045 (1987) (silanes on silica); Maoz and Sagiv, Langmuir 3, 1034 (1987) (silanes on silica); Wasserman et al., Langmuir 5, 1074 (1989) (silanes on silica); Eltekova and Eltekova, Langmuir 3, 951 (1987) (aromatic carboxylic acids, aldehydes, alcohols and methoxy groups on titanium dioxide and silica); Lee et al., J. Phys. Chem. 92, 2597 (1988) (rigid phosphates on metals).

The analyte of interest (typically the nucleic acids of a target bacterium or virus) is contained in the sample to be tested. The term “sample” as used herein refers to any sample that could contain an analyte for detection. The sample may be of entirely natural origin, of entirely non-natural origin (such as of synthetic origin), or a combination of natural and non-natural origins. A sample may include whole cells (such as prokaryotic cells, bacterial cells, eukaryotic cells, plant cells, fungal cells, or cells from multi-cellular organisms including invertebrates, vertebrates, mammals, and humans), tissues, organs, lysates, or biological fluids (such as, but not limited to, blood, serum, plasma, urine, semen, and cerebrospinal fluid). Thus, a sample includes but is not limited to, a cell, a tissue (e.g., a biopsy), the lysates, a biological fluid (e.g., blood, plasma, serum, cerebrospinal fluid, amniotic fluid, synovial fluid, urine, lymph, saliva, anal and vaginal secretions, perspiration, semen, lacrimal secretions of virtually any organism, with mammalian samples being preferred and human samples being particularly preferred). A sample may be an extract made from biological materials, such as from prokaryotes, bacteria, eukaryotes, plants, fungi, multi-cellular organisms or animals, invertebrates, vertebrates, mammals, non-human mammals, and humans. A sample may be an extract made from whole organisms or portions of organisms, cells, organs, tissues, fluids, whole cultures, or portions of cultures, or environmental samples or portions thereof. In addition to the target analyte, in some embodiments the sample may comprise any number of other substances or compounds, as known in the art. In some embodiments, sample refers to the original sample modified prior to analysis by any steps or actions required. Such preparative steps may include washing, fixing, staining, diluting, concentrating, decontaminating, lysis, or other actions to facilitate analysis. A sample may need minimal preparation (for example, collection into a suitable container) for use in a method of the present invention, or more extensive preparation (such as, but not limited to removal, inactivation, or blocking of undesirable material or contaminants, filtration, size selection, affinity purification, cell lysis or tissue digestion, concentration, or dilution).

As discussed below, the nanoparticles serve as carriers with a relatively large surface area to ensure the functionalization of a relatively large quantity of chemiluminescent molecules (and optionally oligonucleotide probes).

In one aspect, the present invention enables the application of nanoparticle-functionalized chemiluminescence for detection of analytes contained in solution. However, the functionalized chemiluminescent nanoparticles are preferably deposited in dry form on a support and may be stored at ambient temperatures. As used herein, “support” is interchangeable with terms such as “solid support,” “solid carrier,” “solid phase”, “surface,” “membrane” or “resin.” All supports comprise at least one surface. Surfaces can be planar, substantially planar, or non-planar.

A support can be comprised of organic polymers such as polystyrene, polyethylene, polypropylene, polyfluoroethylene, polyethyleneoxy, polyacrylamide, polisiloxanes, as well as co-polymers and grafts of any of the foregoing. Some other exemplary support materials include, but are not limited to, latex, polystyrene, polytetrafluoroethylene (“PTFE”), polyvinylidene difluoride (“PVDF”), nylon, polyacrylamide, or poly(styrenedivinylbenzene), or polydimethylsiloxane (“PDMS”). A support can also be inorganic, such as glass, silica, or controlled-pore-glass (“CPG”). The configuration of a support can be in the form of a bead, a sphere, a particle, a granule, a gel, or a membrane. Some non-limiting examples of suitable supports include, but are not limited to, microparticles, nanoparticles, chromatography supports, membranes, or microwell surfaces. Supports can be porous or non-porous, and can have swelling or non-swelling characteristics. Supports can be rigid or can be pliable. A support can be configured in the form of a well, depression, or other container, vessel, feature, or location. A plurality of supports can be configured in an array at various locations, addressable for robotic delivery of reagents, or by detection methods and/or instruments.

Thus, in an exemplary aspect, the present invention is directed to a device comprising a light-shielding container having a fiberoptic cable for transmitting light generated within said light-shielding container to a photon detector. A plurality of functionalized nanoparticles are deposited or captured on or within the support in solid form and placed inside the container. Typically, the functionalized nanoparticles are deposited in a carrier solution, and then the carrier solution is allowed to evaporate leaving the nanoparticles in solid form on or within the support. The area on the support that the nanoparticles are deposited may be the loading region on a lateral flow device (test strip or thin film chromatography).

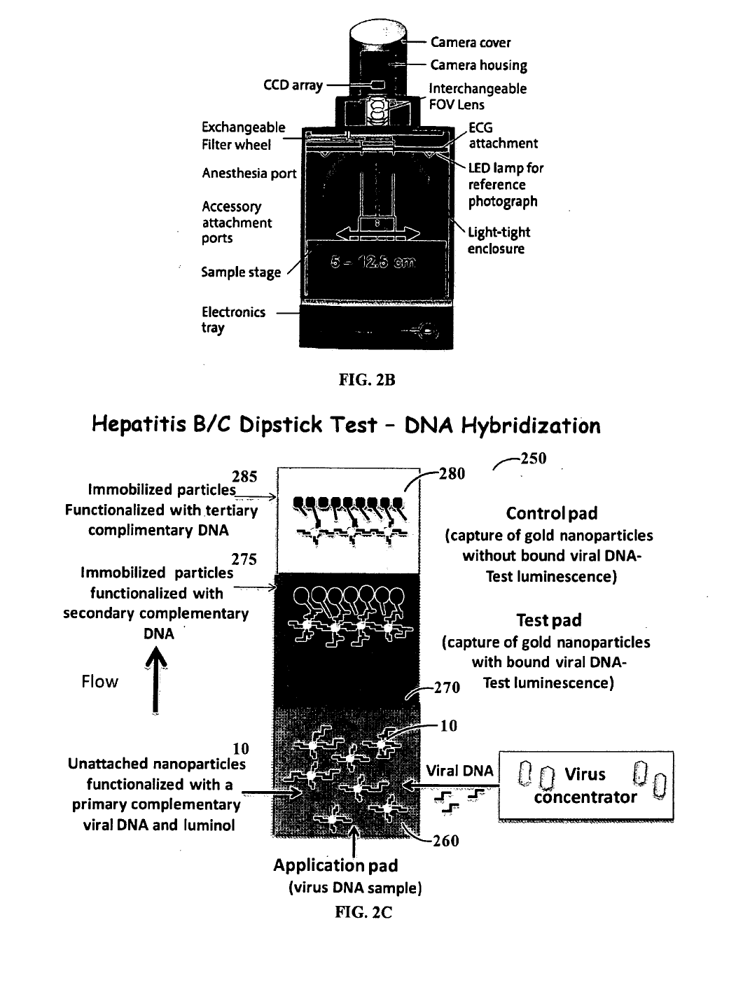

In one aspect, the chemiluminescent compositions of matter of the present invention are used in a multiwell format. An exemplary device is illustrated in panel a of FIG. 2 and FIG. 2B. The device 100 includes a light-shielding container 110 having a fiberoptic cable 120 for transmitting light generated within said light-shielding container to a photon detector 130. A plurality of functionalized nanoparticles 10 are deposited on or within the support in solid form. In this exemplary embodiment, the functionalized nanoparticles 10 (e.g., gold nanoparticles with luminol linked via a carboxylic acid having a terminal thiol group) deposited on or within the wells are directly used to detect analytes (such as red blood cells). During detection, a volume of about 14 to 1 mL of sample 105 is placed in the nanoparticles-containing multiwells. A volume of about 1 μL to 1 mL of reagents which can catalyze the chemiluminescence is dispensed through a microtubing coupled with a syringe or micropipette 160. The chemiluminescent signal is recorded immediately after reagent dispension for up to 5 minutes. For example, luminol reacts with an oxidant (such as hydrogen peroxide) in the presence of a base (such as sodium hydroxide) in the presence of a catalyst (such as a copper(II) or iron(III) catalyst) to produce an excited state product (3-aminophthalate, 3-APA) which gives off light at approximately 425 nm.

More specifically, in panel a of FIG. 2A, the device 100 includes a solid support which is a multiwell plate 150. When the sample 105 is added to one of the wells in the presence of the reagent system 165 (e.g., NaOH and hydrogen peroxide), the presence of iron in any blood in the sample will cause the luminol of the functionalized nanoparticles in the wells to produce light. It will be appreciated that chemiluminescent light typically is transmitted in all directions, and some light will be absorbed or reflected by the walls of the sample holder (well). A substantial portion of the light is transmitted though the top of the well and is collected and transmitted via the fiberoptic cable 120 to the photo detector 130. An exemplary structure for detecting the chemiluminescent light is illustrated in FIG. 2B. The exemplary system is the IVIS Lumina II workstation manufactured by Caliper Inc. which has a light-shield box of 48×71×104 cm (W×D×H) in dimension and encloses a highly sensitive charge coupled device (CCD) as photon detector and optic lens with an adjustable field of view from 5 cm to 12.5 cm. It will be appreciated that the device illustrated in FIG. 2B was used to obtain preliminary data, but that the ultimate design will be much smaller. The dimension of the light shielding container 110 illustrated in FIG. 2A will typically be on the order of about 2×6×4 cm (e.g., a width of about 1, 1.5, 2, 2.5, or 3 cm, a length of about 4, 5, 6, 7, or 8, cm, and a height of about 1, 1.5, or 2 cm).

The nanoparticles of the present invention are also well suited for use in a test strip format based on specific affinity binding between the probe (e.g., oligonucleotide probe) and the analyte (the nucleic acid contents of the target bacterium or virus of interest). An exemplary device is illustrated in panel b of FIG. 2A and FIG. 2C. In such an embodiment, the test strip comprises at least three regions. First, the chemiluminescent-functionalized and probe-functionalized nanoparticles 10 are deposited on an application pad region 260 of the test strip 250. For example, the gold nanoparticles functionalized with both luminol and an oligonucleotide probe capable of selectively hybridizing to the target bacterium or virus (e.g., the X-tail sequence of the Hepatitis C virus) may be placed in solution, deposited on the application pad region 260, and then allowed to dry at room temperature. These functionalized nanoparticles (Probe-CL-NP) are not covalently attached to the pad and are thus mobile. The test pad region 270 contains immobilized nanoparticles 275 (e.g., latex beads) functionalized with a second oligonucleotide probe capable of selectively hybridizing the target bacterium or virus. A control pad region 280 contains immobilized nanoparticles 285 (e.g., latex beads) functionalized with a third oligonucleotide probe capable of selectively hybridizing the first oligonucleotide probe on the mobile chemiluminescent-functionalized nanoparticles 10.

Preferred materials for the test strip pad include thin layer of silica gel, aluminum oxide, or cellulose on glass, plastic, or aluminum foil, etc. Further, similar to the common diagnostic test strips (also called rapid lateral flow test strips), and preferred for water sample, the device may comprise a single strip or a stack of several porous films including paper, nitrocellulose membranes, woven meshes, cellulose filters, thin mats of pre-spun fibers of cellulose, glass, or plastic (such as polyester, polypropylene, or polyethylene).

To immobilize particles on the test strip pads, commercially available membrane sheets (in normal paper size) can be used. Ink printing or a drawing pin can be used to deposit the latex microparticles (or other microparticles such as silica, alumina, iron oxides, etc.) on the testing and control lines. After the solvent is evaporated, the microparticles are left on the membrane surface. The latex beads are covalently attached with the testing probes and control probes, respectively.

In use, the sample is placed before the application pad region 260. As the sample 205 flows through the test pad (typically via capillary action), the bacterium or viral analyte (e.g., the nucleic acids of a Hepatitis C virus) selectively hybridizes to the oligonucleotide probe of the luminol-functionalized nanoparticles 10. The mobile bacterium or virus/Probe-CL-NP hybrid is then captured in the test region 270 by the second hybridization reaction between the nucleic acids of bacterium or virus analyte and the immobilized particles functionalized with the second oligonucleotide probe 275. The immobilized particles functionalized with the third oligonucleotide probe 285 capture the remaining functionalized nanoparticles (Probe-CL-NP) as they flow through the control pad region 280.

When the reagent system 265 (e.g., NaOH and hydrogen peroxide and iron) is added to the test pad region 270 and confined within a blackened elastomer tubing wrapping around the bundle of fiberoptics 220 and reagent-delivery microtubing by pressing the assembly against the test strip, the chemiluminescent moiety of the functionalized nanoparticles 10 will produce light. A substantial portion of the light is collected and transmitted via the fiberoptic cable 220 to the photo detector 230. The assembly is then raised, moved on top of the control pad region 280, and pressed down for similar reagent injection and chemiluminescence reading. If the nucleic acids of the target bacterium or virus are present in the sample, chemiluminescence will be observed in the test pad region 270 because the bacterium or virus/Probe-CL-NP hybrid will be captured in the test pad region 270. In the absence of the nucleic acids of the target bacterium or virus, no chemiluminescence is observed in the test pad region 270. The observation of chemiluminescence in the control pad region 280, however, illustrates that the test pad is working properly since excess Probe-CL-NP will be captured in the control pad region 280.

It will be appreciated that the test strip of the present invention may take a shape of a rectangle, circle, oval, triangle, and other various shapes, provided that there should be at least one direction along which a test solution moves by capillarity. In case of an oval or circular shape, in which the test solution is initially applied to the center thereof, there are different flow directions. However, what is taken into consideration is that the test solution should move in at least one direction toward a predetermined position containing the immobilized second probe. The thickness of the test strip according to the present invention is usually 0.1 to 2 mm, more usually 0.15 to 1 mm, preferably 0.2 to 0.7 mm, though it is not important. In general, a minimum thickness is determined depending on the strength of the strip material, the sorption capability for providing the capillary lateral flow, and needs for producing a readily detectable signal while a maximum thickness is determined depending on handling ease and cost of reagents. In order to maintain reagents and provide a sample of a defined size, the strip is constructed to have a relatively narrow width, usually less than 20 mm, preferably less than 10 mm. In general, the width of the strip should be at least about 1.0 mm, typically in a range of about 2 mm to 12 mm, preferably in a range of about 4 mm to 8 mm.

The test strip may also include a backing (not shown). The backing is typically made of water-insoluble, non-porous, and rigid material and has a length and width equal to the pads situated thereon, along which the sample develops, but may have a dimension being less or greater than the pad. In preparation of the backing, various natural and synthetic organic and inorganic materials can be used, provided that the backing prepared from the material should not hinder capillary actions of the absorption material, nor non-specifically bind to an analyte, nor interfere with the reaction of the analyte with a detector. Representative examples of polymers usable in the present invention include, but are not limited to, polyethylene, polyester, polypropylene, poly(4-methylbutene), polystyrene, polymethacrylate, poly(ethylene terephthalate), nylon, poly(vinyl butyrate), glass, ceramic, metal, and the like. On the backing, a variety of pads are adhered by means of adhesives. Proper selection of adhesives may improve the performance of the strip and lengthen the shelf life of the strip. According to the present invention, pressure-sensitive adhesives (“PSA”) may be representatively used in the lateral flow assay strip. Typically, the adhesion of different pads of the lateral flow assay strip is accomplished as the adhesive penetrates into pores of the pads, thereby binding pads together with the backing.

The application pad region 260 basically acts to receive the fluid sample containing an analyte. It includes the unimmobilized chemiluminescent-probe-labeled nanoparticles 10 for selectively hybridizing to the bacterium or virus of interest in the sample 205. The material in the application pad region 260 preferably had a rapid filtering speed and a good ability to hold particles. As such, synthetic material such as polyester and glass fiber filter can be used. Other materials include paper, cotton, polyester, glass, nylon, mixed cellulose esters, spun polyethylene, polysulfones, and the like. Preferably, nitrocellulose, nylon, or mixed cellulose esters are used for the analyte detection membrane strip 12. Methods for depositing the functionalized nanoparticles onto the application pad region 260 include an impregnation process in which a pad such as glass fiber is immersed in a solution of the functionalized nanoparticles reagent particularly formulated, followed by drying. The functionalized pads on the control region 250 and test pad regions 270 may be deposited using inkjet printing methods.

Now, the present invention will be described in detail using embodiments shown in the following examples. However, the examples are for illustration of the present invention and do not limit the scope of the present invention thereto.

### EXPERIMENTAL

In the following examples, citrate protected GNPs (8.0-12.0 nm in diameter), and mercaptoundecanoic acid were purchased from Sigma Aldrich. Luminol (“LUM”), 1-ethyl-3-(3-dimethylaminopropyl) carbodiimide hydrochloride, N-hydrosuccinimide, Tween 20, potassium ferricyanide (K₃Fe(CN)₆), phosphate buffer saline (“PBS”), sodium hydroxide, and hydrogen peroxide (H₂O₂) were obtained from Fisher Scientific. Polydimethylsiloxane (“PDMS”) was ordered from Dow Corning. All chemicals used in this study were analytical grade. Deionized (“DI”) water with a resistivity of 18.2 MΩ-cm from a portable filtration system (Easy Pure II, Milipore) was used in all the experiments.

For the blood sample preparation, whole sheep blood was obtained from HemoStat Laboratories (Dixon, Calif.). The concentration of the sheep red blood cells in the stock blood solution was measured as about 4.6×10⁹ cells/ml using Petroff Hausser counting chamber under an upright optical microscope (AxioSkop II, Carl Zeiss). The received blood sample was stored at about 4° C. Before chemiluminescent experiments, the sample was inspected under an optical microscope, to make sure that the cells were intact. In the experiments using lysed cells, 100 μL blood samples were frozen at −20° C. and thawed on ice before use. This resulted in complete cell lysis.

UV-visible absorption spectra were recorded using Beckman DU640 spectrophotometer in a 360 μL microcuvette with an optical path length of 10.0 mm. Infrared spectroscopy (“IR”) was performed on a Nicolet 380 FT-IR spectrophotometer with neat solid samples in transmission mode. Transmission electron microscopy (“TEM”) measurements were carried out using FEI Tecnai F20 XT field emission system.

Chemiluminescent experiments were carried out using a IVIS Lumina II system (Caliper Life Sciences, CA), which utilizes a highly sensitive, −90° C. cooled, and back illuminated CCD camera as the detector. A layer of PDMS of about 1.5 mm in thickness was laid on a glass microscope slide (3″×1″×1 mm) in which an array of oval shaped holes (3 mm×4 mm) was punched through to form chemiluminescent reaction wells of about 12 μl in volume. The GNP-MUA-LUM solution was dropped in the well and dried before chemiluminescent measurements. Each well contained a known number of luminol modified GNPs. Typical chemiluminescent experiments involved mixing 4 μl of 0.033 M NaOH, 4 μl of 0.47 M H₂O₂, and 4 μl of 1 mM Fe(CN)₆³⁻ solution or, in some experiments, blood samples (at varied concentrations) in different PDMS wells. The slide was then quickly placed in the light tight black box of the IVIS Lumina II system. A bright field reference photograph was first recorded using the CCD camera (this process takes about 3 seconds), and then the chemiluminescent signal (Photon flux) was recorded in the kinetic mode (i.e., flux of photons vs. time) with an exposure time of 10 seconds to the CCD camera. The chemiluminescent signal is represented in a pseudocolor image by overlaying the bright-field and chemiluminescent images. The elapse between consecutive chemiluminescent snap shots in the kinetic mode is approximately 13 seconds (i.e., 3 seconds for reference photograph, and 10 seconds to collect chemiluminescent signal). Normally, 10 such chemiluminescent snapshot images were taken and the integrated photon flux over the designated PDMS well was plotted vs. time.

### Example 1

### Modifying GNPs with Chemiluminescent Luminol

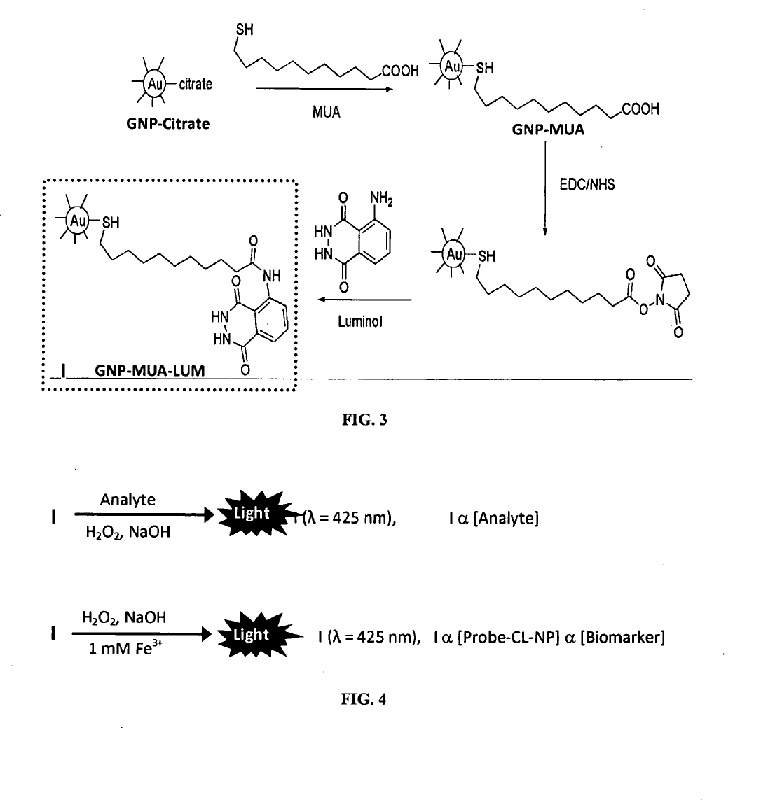

In this example, GNPs were functionalized with a chemiluminescent material according to the scheme outline in FIG. 3. The first step was to exchange the citrate groups with the MUA ligand on the surface of GNPs under the protection of the nonionic surfactant Tween-20. Typically, 2 ml of citrate-protected GNP stock solution (5.99×10¹¹ particles/ml) was transferred in a clean, dry test tube with a screw-cap followed by addition of 2 ml of 1×PBS with 0.2 mg/ml Tween-20 buffer (the same buffer composition was used for all following steps during functionalization). The mixed solution was incubated at room temperature (“RT”) for 30 minutes before 2 ml of 3.0 mM MUA solution (in 1:3 ethanol/DI water) was added. The solution was further incubated overnight at RT with gentle shaking. The mixture was centrifuged at 14,100 rpm for 20 minutes to pellet the MUA-covered GNPs (GNP-MUA). The supernatant was discarded and the pellet was re-suspended in the buffer. The pellet was washed three more times before the final suspension in the buffer. MUA modified GNPs (200 μl) were then reacted with 100 μl of freshly prepared aqueous solution of 50 mM EDC and 50 mM NHS for 15 minutes. This mixture was then combined with 100 μl of 50 mM LUM solution (a few drops of 0.4 M NaOH were added to increase the solubility of LUM in DI water) and incubated at RT for 2 hours. Finally, the LUM-modified GNPs (GNP-MUA-LUM) were washed 3 times with buffer and finally suspended in the buffer solution to obtain a final concentration of about 1×10¹² GNP/ml.

In sum, the two-step strategy to functionalize luminol on GNPs and the scheme of using such functionalized GNPs for detecting Fe³⁺ containing analytes are illustrated in FIG. 3. In the first step, the citrate ligand, which was used to stabilize GNP colloid in the starting materials was replaced with MUA by ligand exchange. This process produced a self-assembled monolayer of MUA on each GNP through stronger Au-thiol interaction, yielding carboxylic acid (—COOH) terminal groups at the exterior surface. In the second step, the MUA derivatized GNP colloid was reacted with luminol in the presence of EDC and NHS, which facilitated the covalent binding of luminol onto the GNPs via an amide bond formed between the —COOH group of MUA and the —NH₂ group of luminol. The product is labeled as compound I (i.e., GNP-MUA-LUM) in FIG. 3. UV-vis, IR, and HRTEM measurements were employed at each stage of modification to confirm physical and chemical changes occurring at the surface of the GNPs.

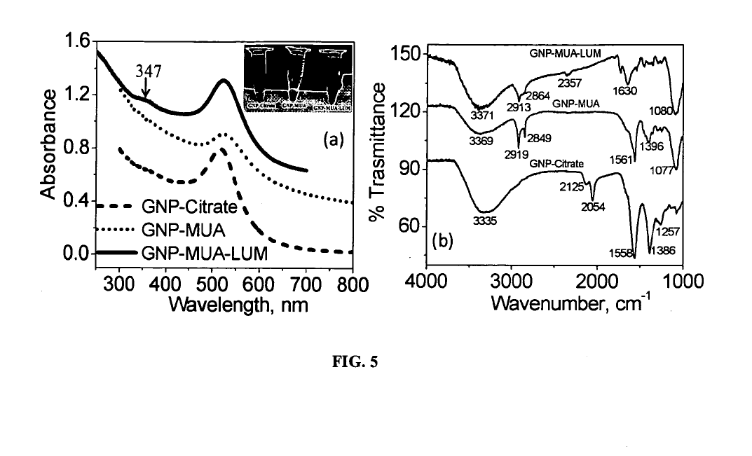

The UV-visible absorption spectra in FIG. 5 (panel a) show the GNPs with different functional moieties at each stage, i.e., GNP-citrate, GNP-MUA, and GNP-MUA-LUM. Strong absorption peaks were observed for all GNPs at about 516 nm, corresponding to the SPR. The full-width-half-maximum in the case of GNP-MUA and GNP-MUA-LUM is slightly larger than that of the GNP-citrate. The wavelength at the peak absorption of the GNP solution remains the same (as indicated by the deep red color shown in inset of FIG. 5). These data indicate that the particle size remains similar as it goes through the ligand exchange and luminol functionalization processes. For GNP-MUA-LUM, however, there is an additional small, but noticeable peak at 347 nm, which corresponds to one of the absorption peaks of the luminol.

FIG. 5 (panel b) shows the FT-IR spectra of neat solid GNPs at different steps of functionalization. For MUA modified GNPs, the characteristic IR absorption peaks can be clearly seen at 2919 and 2849 cm⁻¹, which can be ascribed to the vibrational stretches of —CH₂— functional groups in the MUA chain. A peak corresponding to the C═O stretch in the terminal carboxylic acid group of MUA is expected at about 1700 cm⁻¹, but it was shifted to about 1550 to 1610 cm⁻¹ for GNP-MUA and split between 1600 to 1730 cm⁻¹ for GNP-MUA-LUM. This indicates that the carboxylic acid group in GNP-MUA presents in the ionized form (i.e., as carboxylate salts) since the pH value of the suspension solution is about 7 above the pKa of general —COOH groups. The IR absorption at 1600 to 1730 cm⁻¹ in GNP-MUA-LUM is consistent with the formation of amide bonds between the —COOH group in MUA and the —NH₂ group in luminol. Also, a peak at 1396 cm⁻¹ corresponding to the bending of C—H bond in the long alkane chain can be seen in GNP-MUA. The peaks corresponding to the C—H stretch of —CH₂— in the alkane chain were observed at 2913 and 2864 cm⁻¹ in GNP-MUA-LUM, confirming that the MUA monolayer was intact after LUM functionalization. The N—H stretch mode of luminol, which is expected to be at 3,300 to 3,500 cm⁻¹, however, was buried under the strong background absorption by GNPs. Overall, the FTIR spectra confirmed that the ligand exchange to replace citrate with MUA and functionalization of LUM to MUA were successful following the schemes shown in FIG. 3.

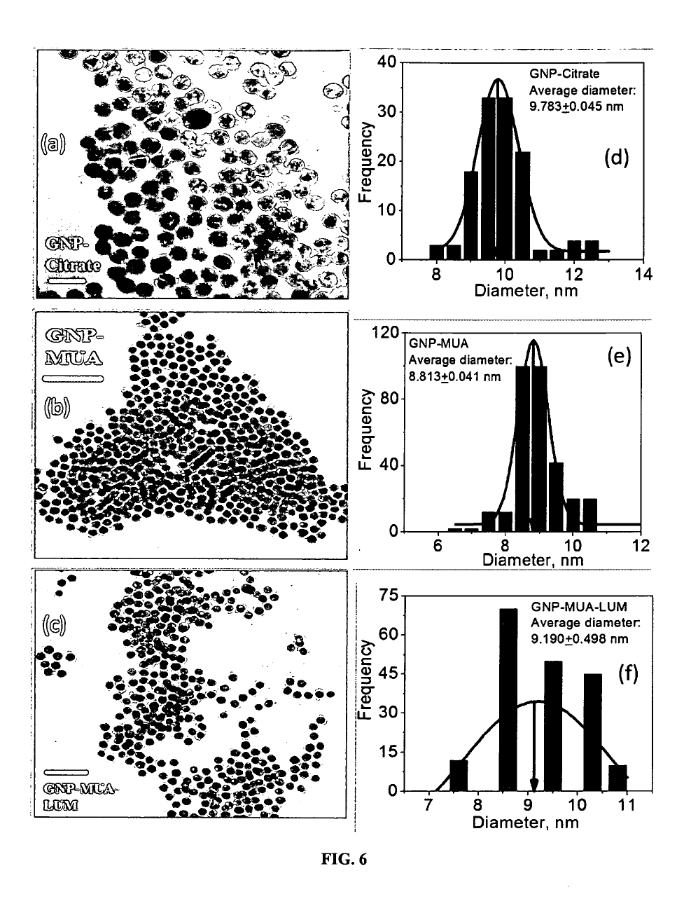

TEM images in FIG. 6 further confirm that the shape and size of the GNPs before and after the modification have not been altered. The average diameter of citrate-stabilized GNPs was found to be about 9.78±0.05 nm, in good agreement with the average size of 10 nm and a range distribution between 8.0 and 12.0 nm as certified by the vendor. After ligand exchange and luminol functionalization, the measured size of GNPs changed to about 8.81±0.04 nm and about 9.2±0.5 nm, respectively, within the size range of 8.0-12.0 nm, and no noticeable aggregation was observed.

### Example 2

### Chemiluminescent Assessment

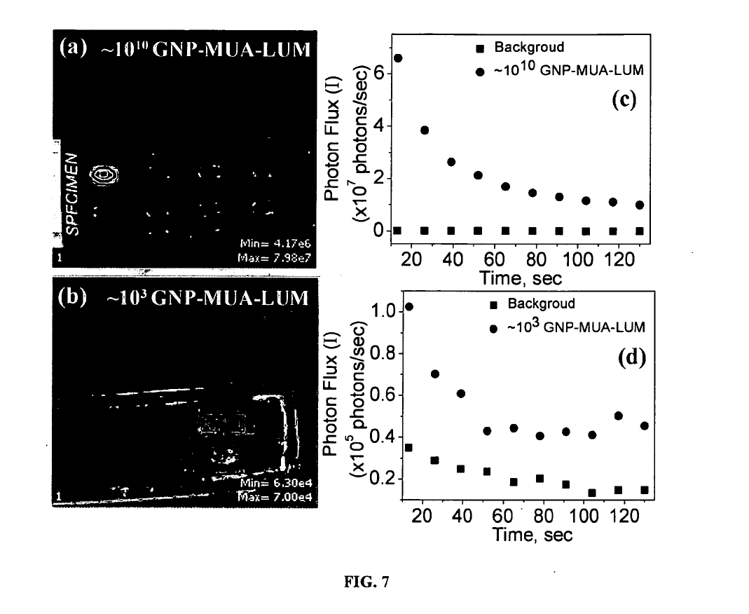

After functionalizing GNPs with chemiluminescent luminol molecules, the concentration of the stock solution was adjusted such that a 10 μl solution dispensed about 1×10¹⁰ GNPs. This was used in a series of dilutions to obtain GNP-MUA-LUM solutions at concentrations varying over 8 orders of magnitude. The PDMS wells on the test support were loaded with a 10 μl solution of respective concentration and dried in the incubator before chemiluminescent measurements. FIG. 7 (panels a and b) includes representative snapshot CCD images of chemiluminescent signals recorded during the chemiluminescent measurements from the PDMS wells loaded with 1×10¹⁰ and 1×10³ GNP-MUA-LUM, respectively. Photons were emitted immediately upon addition of the premixed solution consisting of 4 μl of NaOH (0.033 M), 4 μl of H₂O₂ (0.47 M), and 4 μl of Fe(CN)₆³⁻ (1.0 mM) to the PDMS wells. The region of interest in the image was selected over the specific PDMS well using the IVIS Lumina II system software and the photon counts was integrated over this region.

It will be appreciated that the mechanism of light production by luminol in different solvents has been previously explored by several researchers. The chemiluminescent reaction of luminol generally utilizes Fe³⁺ as catalyst and requires two equivalents of base to deprotonate the nitrogen protons, leaving a negative charge which then undergoes resonance to form an enolate ion. Then a cyclic addition reaction of the oxygen at the two carbonyl carbons takes place with the oxygen provided by peroxide (with Fe³⁺ catalyzing the breakdown of peroxide into oxygen and water), leading to the expulsion of N₂ in the gaseous form. This step leads to the formation of 3-aminophthalate (an excited form of luminol) and light emission peaked at the wavelength of μₘₐₓ=425 nm while electrons return to the ground state. Chemiluminescence of luminol is known to follow a flash mechanism in which chemiluminescence occurs immediately and then decays quickly. The half-life strongly depends on the experimental conditions. It can be seen in FIG. 7 (panels c and d) that the integrated chemiluminescent signal (filled circles) has the maximum value at the first snapshot image for the PDMS wells loaded respectively with 1×10¹⁰ to 1×10³ luminol labeled GNPs. The data sampling rate was limited by the imaging speed at about 13 second/frame. The chemiluminescent signal decayed exponentially with time as shown in FIG. 7 (panels c and d). Nevertheless, the chemiluminescent signal from 1×10³ luminol-labeled GNPs clearly remained above the background (filled squares) which was recorded by replacing the 1.0 mM Fe(CN)₆³⁻ solution with DI water while all other experimental settings were kept the same. The half life is about 30 seconds for both 1.0×10¹⁰ to 1.0×10³ luminol labeled GNPs, indicating that the chemiluminescent mechanism remained the same over such a large range.

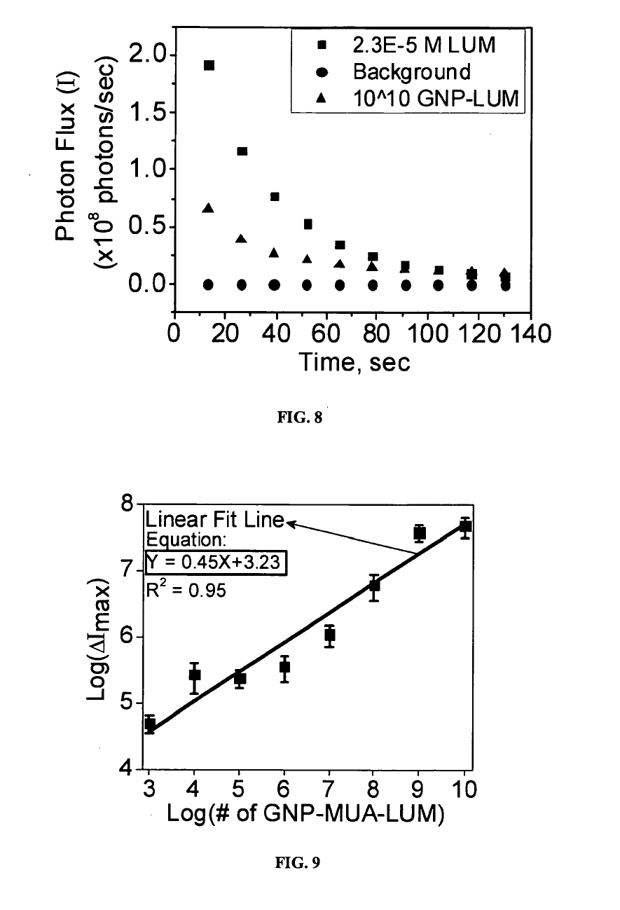

In the experiment with the lowest number of luminol labeled GNPs (i.e., about 1,000 GNPs), the total number of chemiluminescent photons was comparable to the estimated number of luminol molecules (about 1.4×10³ luminol/GNP) by assuming the formation of a close-packed thiol monolayer with the same density as on a flat gold surface. But the large variation in the measurement value limited the assessment of exact value of chemiluminescent quantum yield of the attached luminol molecules. In an alternative approach, the chemiluminescent signal measured with 1.0×10¹⁰ luminol-labeled GNPs was compared with that from the same number of free luminol molecules that were dispersed in solution (4 μL of 23 μM of luminol in each PDMS well) with all other parameters the same. As shown in FIG. 8, the maximum chemiluminescent signal from GNP-MUA-LUM is about 37% of that from the luminol solution. The reduction factor is about 2.7, much smaller than the 5.0 times reduction in the previous study by Yang (2010) using 30 nm diameter GNPs through a much shorter linker (3-mercaptopropionic acid). If the absorption of the chemiluminescent photons by GNPs is considered, the difference between luminols attached to GNPs and those freely dispersed in solution in the measurements is even smaller. This is probably why ultrahigh sensitivity was obtained in this study.

Due to the fast decay in the chemiluminescent signal, it is preferable to use the signal from the first snapshot (i.e., the maximum chemiluminescent signal Iₘₐₓ) instead of the average signal for quantitative analyses. FIG. 9 shows a calibration curve in which the background subtracted maximum chemiluminescent signal (ΔIₘₐₓ) is plotted vs. the number of luminol-labeled GNPs in a PDMS well. A linear relationship between the chemiluminescent signal and the number of GNPs was obtained from 1×10³ to 1×10¹⁰ GNPs as

Log(ΔIₘₐₓ)=0.45 Log(N_{GNP})+3.23 (1)

with an R² value of 0.95, where N_{GNP} is the number of GNPs placed in the PDMS well. Even though chemiluminescent signal from 1,000 GNPs can be clearly observed with ΔIₘₐₓ=about 5.0×10⁴ photons/s (see FIG. 7, panel d), the rigorous statistical detection limit depends on the standard deviation of the chemiluminescent measurements with blank samples (with s_{blank}=1.9×10⁴ photons/s). Following the convention, the signal at the detection limit needs to be:

I_{DL}=I_{blank}+3s_{blank} (2)

where the background signal I_{blank} is about 2.4×10⁴ photons/s. Therefore, the statistical detection limit is derived to be about 2,600 GNPs. This can be improved by reducing the variation of the background reading which was due to the variation in the experimental setting and the drift of the CCD camera.

The chemiluminescent signal should be, in principle, proportional to the concentration of the luminol. However, the relationship between the background-subtracted maximum chemiluminescent signal (ΔIₘₐₓ) and the number of luminol-attached GNPs (N) was ΔIₘₐₓ∝N^{0.45} instead of a linear relationship as ΔIₘₐₓ∝N. This might be due to luminol molecules being attached to the surface of GNPs which were deposited at the bottom of the well. It is a pseudo-two-dimensional system instead of the usual dispersion in bulk solution. The mechanism is in further investigation.

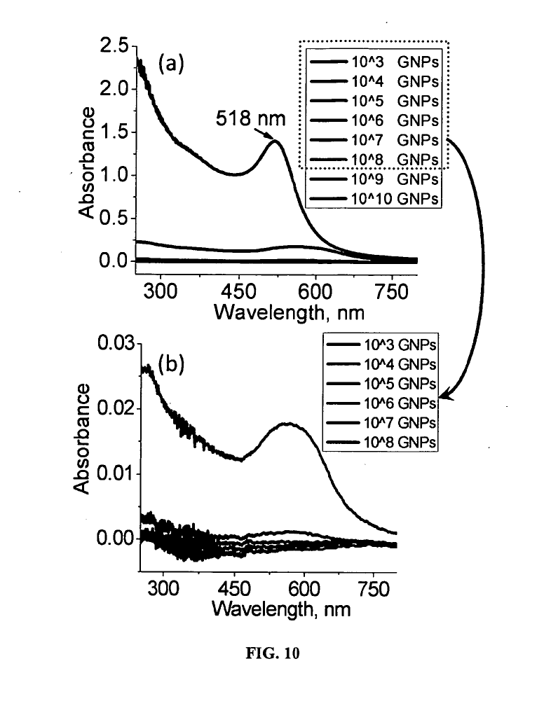

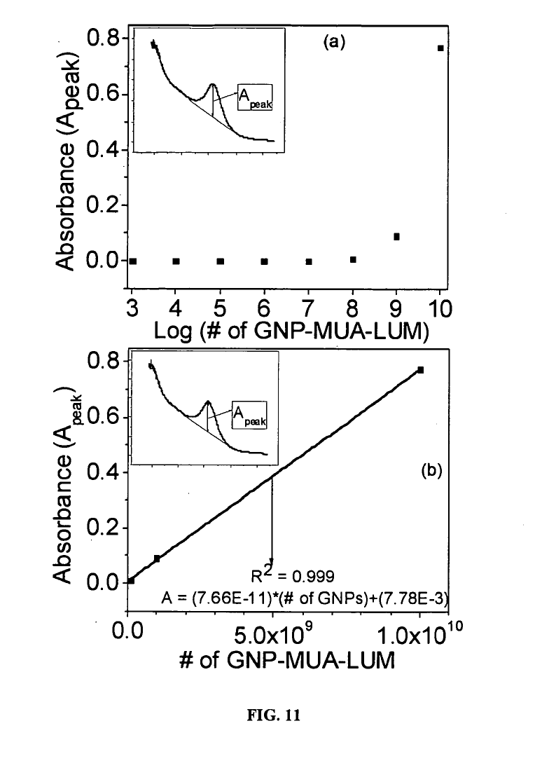

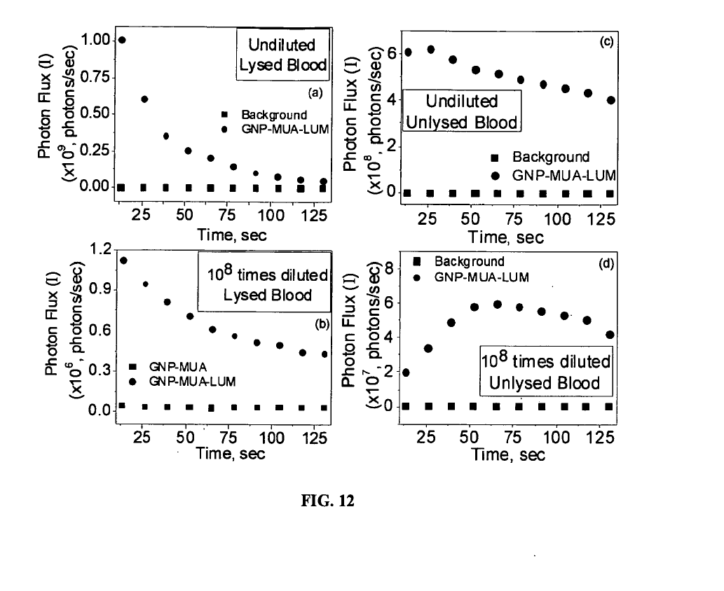

GNPs are known to present strong surface plasma resonance (“SPR”), which has been widely utilized to enhance the sensitivity in colorimetric or optical absorption methods. The results suggest that chemiluminescence can provide even higher detection sensitivity. To compare chemiluminescent with absorption approaches, FIG. 10 shows the UV-visible absorption spectra of GNP-MUA-LUM measured with 350 μl solution in a microcuvette of 10.0 m optical path length. The total number of GNPs is varied from 1×10¹⁰ to 1×10³. At high concentrations (≧ about 1×10⁸ GNPs), it shows a strong absorption peak at 518 nm, corresponding to the SPR of GNPs of about 10 nm in diameter. However, the absorption is below the baseline noise as the number of GNPs is at or below 1×10⁷. Also, the red color associated with the GNPs is only visually observable with naked eyes when the number of GNPs is more than about 1×10⁹. The height of the absorption peak at 518 nm is fitted and plotted against the number of GNPs in the solution in FIG. 11. Clearly, the peak absorbance varies linearly with the number of GNPs when it is near or above 1×10⁸, but quickly drops below the detection limit when it is less than 1×10⁸. In contrast, the chemiluminescent signal using luminol-labeled GNPs can be easily observed with as few as 1,000 GNPs (FIG. 7, panel d)

### Example 3

### Chemiluminescent Detection of Unlysed and Lysed Red Blood Cells

As illustrated in FIG. 4, the luminol-labeled GNPs can be used for chemiluminescent detection under two different schemes. This example focused on demonstrating the detection of blood samples using Scheme I (in which the analyte is blood). Unlysed and lysed sheep blood samples were used to replace Fe(CN)₆³⁻ ions as the analyte which also serves as the catalyst to generate luminol chemiluminescent. The solutions containing about 1×10¹⁰ luminol-labeled GNPs were preloaded in different PDMS wells and the solvent was then dried out. Chemiluminescent measurements were performed after adding the mixture of 4.0 μl of NaOH (0.033 M) and 4.0 μl of H₂O₂ (0.47 M) as well as 4.0 μl of blood sample at desired concentrations. The concentration of the sheep red blood cells in the stock blood solution was about 4.6×10⁹ cells/ml by cell counting. The size of the sheep red blood cell is about 3 to 4 μm. In some experiments, the sheep red blood cells were lysed following the procedure described above. The representative kinetic chemiluminescent data obtained with the stock solutions of unlysed and lysed blood samples, respectively, and with those after 10⁸ times dilution are shown in FIG. 12. The chemiluminescent signal of the lysed blood samples experienced a rapid decay with a half life of about 30 seconds (FIG. 12), similar to what was observed with Fe(CN)₆³⁻ ions (as shown in FIG. 7, panels c and d). It is remarkable that such a strong chemiluminescent signal can be observed with the lysed blood samples even after dilution by 10⁸ times, which corresponds to about 0.18 cell/well.

Interestingly, the unlysed blood samples showed quite different kinetics in chemiluminescent measurements in both original and diluted samples. As shown in FIG. 12 (panels c and d), the chemiluminescent signal rises in the initial period (about 26 and 65 seconds, respectively) and then slowly decays. The rising and decay rates were lower in the highly diluted sample as compared to the original one. This is likely because the red blood cells need to be lysed first to release the hemoglobin to the exterior environment. The degradation of the polypeptidic portion of the hemoglobin then takes place, removing the protection to the reduced form of iron (i.e., Fe²⁺) at the center of the histidine coordination. As a result, Fe²⁺ is quickly oxidized into Fe³⁺ and becomes an active catalyst to facilitate the reaction of luminol molecules to generate chemiluminescence. In the stock solution of the unlysed blood sample, there are likely many residual hemes outside the cell, hence the initial rise in chemiluminescent signal is not prominent. But for the sample diluted by about 10⁸ times (to about 46 cells/ml), likely only a single red blood cell is randomly picked and dispensed into the PDMS well, which was lysed by the high concentration of NaOH (about 0.01 M after mixing) to release hemoglobin for subsequent chemiluminescent reaction. Hence, the generation of chemiluminescence is delayed by about 65 seconds.

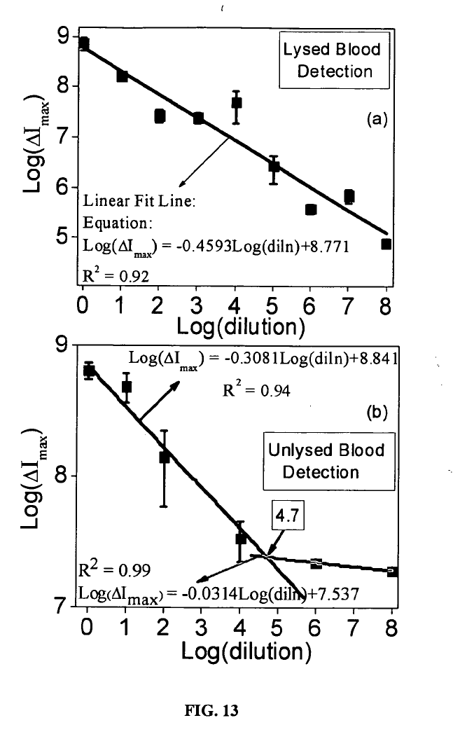

FIG. 13 shows the log-log plots of the background subtracted maximum chemiluminescent signal (ΔIₘₐₓ) vs. the dilution factor for lysed and unlysed blood samples, respectively. A linear relationship between log(ΔIₘₐₓ) and log(dilution) was obtained for the lysed sample in a large range of the dilution factor ranging from 0 to 10⁸. A slope of −0.459 is obtained from FIG. 13 (panel a), which is very close to that of log(ΔIₘₐₓ) vs. log(N_{GNP}) (with N_{GNP} as the number of luminol-labeled GNPs) in FIG. 9. This confirms that the chemiluminescence in these experiments is likely based on the same mechanism (i.e., Scheme 1 in FIG. 4). The unlysed blood sample in FIG. 13, panel b, however, shows a transition at the dilution factor of about 5×10⁴. Two straight lines are needed to fit the experimental data, with a slope of −0.308 below 10⁴ times dilution and a very small slope of −0.031 above 10⁵ times of dilution. At the transition point of a dilution factor of about 5×10⁴, there are about 370 cells dispensed in the PDMS well by calculation. This number is close to the limit of statistically reliable sampling. Other catalysts beside the hemoglobin from the intact red blood cells may also contribute to the chemiluminescent signal and generates the chemiluminescence even after 10⁸ times dilution even though the slope is much smaller.

In short, the foregoing illustrates that the preparation of luminol-functionalized gold nanoparticles with convincing characterization with UV-Vis and IR spectroscopy and transmission electron microscopy. In a preliminary test, luminol-functionalized gold nanoparticles were exposed to blood samples of different concentrations to determine the detection sensitivity which exceeded that of conventional colorimetry assay by about 5 orders of magnitude. It also improves the detection limit of conventional solution-based chemiluminescence by at least 3 orders of magnitude. With the enhancement in signal, detection of blood samples after dilution by 10⁸ times was made—down on single red blood cells.

### Example 4

### Comparison of Sensitivities of Luminol in Bulk Solution vs. Luminol-Labeled GNPs

In this example, the comparison of chemiluminescence signal of luminol molecules in bulk aqueous solutions and equivalent amount of luminol molecules covalently attached to 10 nm diameter gold nanoparticles was made. The number of luminol on each GNP (d=10 nm) was calculated by assuming a close-packed monolayer at a density of 5.0×10⁴ luminol/cm2 on the outer surface (πd²) of each GNP, giving 1.6×10³ luminol/GNP. The concentration of GNPs was varied over many orders of magnitude in these measurements. The straight lines are linear fitting of the chemiluminescence signal (above the background) vs. the luminol concentration in log-log scale.

Chemiluminescence Measurement Conditions:

Chemiluminescence experiments were carried out using luminescent mode from GloMax-Multi+ Microplate Multimode Reader. Round bottom 96 wells white polystyrene plate was used in all the luminescent measurements. In the luminol bulk solution experiment, 25 μL of 0.1 M NaOH, 25 μL of 1.408 M H₂O₂ and 25 μl, of 1 mM K₃Fe(CN)₆ solution were preloaded in one well of the 96 well plate. Then 25 μL of luminol solution in varying concentration (10⁻¹⁴ to 10⁻⁵ M) was added by the injector from the instrument into the above mixed solution to initialize the chemiluminescence reaction. The injection speed is 200 μL/sec. The chemiluminescence signal was recorded for about 8 minutes after injection of the reagents. In the GNP-MUA-LUM solution experiment, 25 μL of 0.1 M NaOH, 25 μl of 1.408 M H₂O₂ and 25 μL of GNP-MUA-LUM solution in varying number of the GNPs (1.82×10² to about 1.82×10¹⁰ GNPs) were preloaded in the 96 well plate. Then 25 μL of 1 mM K₃Fe(CN)₆ solution was added into the mixture solution by the injector to start the reaction. The chemiluminescence signal was recorded for about 8 minutes after injection of the reagents. In both experiments, the background signal (after 8 minutes) was deducted from the highest chemiluminescence signal (i.e., the first data point) to give ΔI which was used as the corrected signal for each measurement.

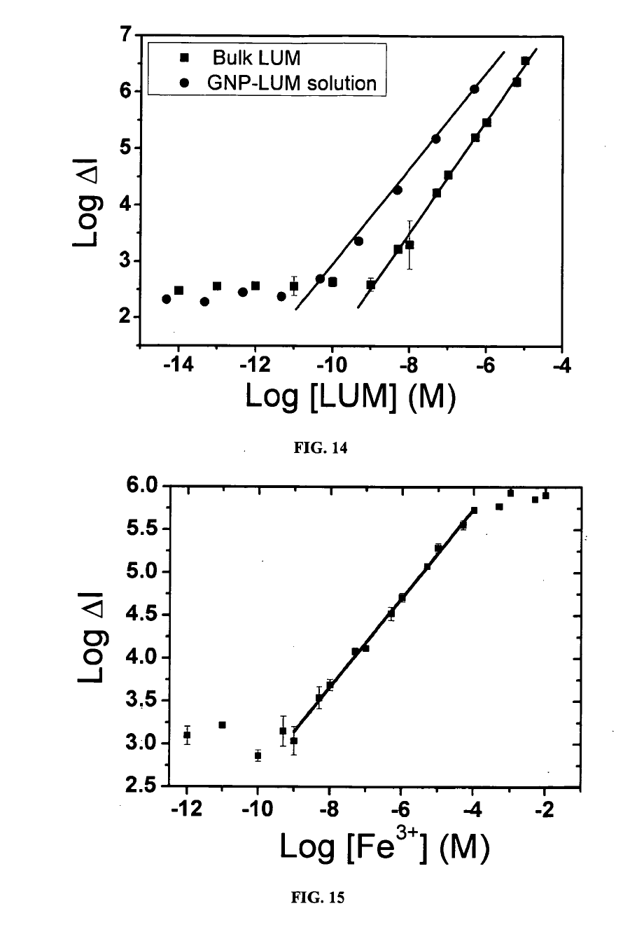

As shown in FIG. 14, from the intersection of the linear fitting line and the flat background baseline, the detection limit can be derived as about 1×10⁻⁹ M for bulk luminol solutions and about 3×10⁻¹¹ M equivalent concentration for LUM-GNPs. By the same method, the detection limit of equivalent luminol concentration in LUM-GNP can be derived as about 3.0×10⁻¹¹ M. This is translated into about 2.0×10⁻¹⁴ M of LUM-GNPs (with about 1.6×10³ luminol/GNP).

The foregoing results can be used to extrapolate the detection limit of the invented test strip. The current instrument only measures a small portion of the LUM-GNPs in the 100 μL volume. With the inventive devices, the detection efficiency can be increased by a factor of at least 100 on the test strip. Thus, the detection limit in terms of number of LUM-GNPs is (about 2.0×10⁻¹⁴ M)×(100×10⁻⁶ L)×(6.03×10²³/mole)/100=about 6.8×10³. In principle, about one target nucleic acid is needed to capture on LUM-GNP onto the test strip. Thus, detection down to about 10,000 copies of virus DNAs or RNAs can be made. With further optimization with larger GNP and chemiluminescence enhancers, the detection limit can be further reduced to about 1,000. This is sufficient for detecting virus or bacterial without PCR amplification.

### Example 5

### Fe³⁺ Detection

In this example, as shown in FIG. 15, a calibration curve of chemiluminescence signal vs. Fe³⁺ catalyst concentration in bulk luminol solutions was prepared. The straight lines are linear fitting of the chemiluminescence signal (above the background) vs. Fe³⁺ concentration in log-log scale. A dynamic range of about 5 orders of magnitude can be obtained.

The chemiluminescence experiments were carried out using luminescent mode from GloMax-Multi+Microplate Multimode Reader. Round bottom 96 wells white polystyrene plate was used in all the luminescent measurements. At first, 25 μL of 0.1 M NaOH, 25 μL of 1.408 M H₂O₂ and 25 μL of K₃Fe(CN)₆ solution at varied concentrations were preloaded in the wells of a 96 well plate. Then 25 μL of luminol solution at 1 mM concentration was added by the injector into the above mixed solution to initialize the chemiluminescence reaction. The injection speed was 200 μL/sec. The chemiluminescence signal was recorded for about 8 minutes after injection of the reagents. The background signal (after 8 minutes) was deducted from the highest chemiluminescence signal (i.e., the first data point) to give DI which was used as the corrected signal for each measurement.

The foregoing illustrates that the dynamic range for Fe³⁺ detection using chemiluminescence spanned about 5 orders of magnitude from 1×10⁻⁹ M to 1×10⁻⁴ M. The detection limit for Fe³⁺ is about 1×10⁻⁹ M. Thus, a single red blood cell using chemiluminescence may be detected.

### Example 6

### Selection of Suitable Chlamydia Specific Oligonucleotides

In this example, Chlamydia specific oligonucleotides were selected based upon unique open reading frames (ORFs) identified in a large-scale comparative genomic analysis. “BLAST screening can be used chlamydial genomes to identify signature proteins that are unique for the Chlamydiales, Chlamydiaceae, Chlamydophila and Chlamydia groups of species” Table 4 of Griffiths et al. BMC Genomics (2006), which is incorporated by reference, identified Chlamydia trachomatis specific proteins. These proteins are uniquely found in species belonging to the Chlamydia genus and are absent in Chlamydophila and Protochlamydia.

The DNA sequence of these proteins were used to search the existing NCBI database and identify regions of 100% DNA sequence identity within Chlamydia trachomatis. Sequence were blasted again using somewhat dissimilar sequences to eliminate any human matching sequences (or expected human associated organisms).

The following sequence is from ORF CT135 and is 100% identical for all deposited Chlamydia trachomatis DNA sequences (nt 63-127).

SEQ ID NO 10:
TCGCATGCTCAATAGTGCGACTTGTGCTGCTGGCGGCATAGGA
TTGTTAACACCAGTGGTATGC (64 mer)

The following sequence is from ORF CT326.2 and is 100% identical for all deposited Chlamydia trachomatis DNA sequences.

SEQ ID NO 11:
ATGAACACACTCAGTTTTAGAAACGCTTTTG (31 mer)

The following sequences is from ORF 115 and is 100% identical for ALL deposited Chlamydia trachomatis DNA sequences.

SEQ ID NO 12:
GGCAGTTGCTGTGGCCACTATATTGGCC (28 mer)
SEQ ID NO 13:
TAGCGGCATCTTTATTCTTCGGGGTAGG (28 mer)
SEQ ID NO 14:
TTGGAGGAGTGCTGACTACAGAAGCTGTGA (30 mer)
SEQ ID NO 15:
CATCGATCACAAACTTTGATGTGGAACAACTTATGCTGTAAAAC
(44 mer)
SEQ ID NO 16:
GCAGAGGTTGAGCAGAAAATCTCGACAGCTAGTGCAAATGCC
AAAAGCAATGATAAG (57 mer)

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

All publications and patent applications mentioned in this specification are herein incorporated by reference to the same extent as if each individual publication or patent application was specifically and individually indicated to be incorporated by reference.
Abad et al., Functionalization of Thioctic Acid-Capped Gold Nanoparticles for Specific Immobilization of Histidine-Tagged Proteins, J Am Chem Soc 127 (15) 5689-5694 (2005).
Alivisatos et al., Organization of ‘nanocrystal molecules’ using DNA, Nature 382 (6592) 609-611 (1996).
Baptista et al., Gold nanoparticles for the development of clinical diagnosis methods, Analytical and Bioanalytical Chemistry 391 (3) 943-950 (2008).
Barni et al., Forensic application of the luminol reaction as a presumptive test for latent blood detection, Talanta 72 (3) 896-913 (2007).
Bartz et al., Monothiols derived from glycols as agents for stabilizing gold colloids in water: Synthesis, self-assembly, and use as crystallization templates, J Mater Chem 9 (5) 1121-1125 (1999).
Bergervoet et al., Application of the forensic Luminol for blood in infection control, J Hosp Infect 68 (4) 329-333 (2008).
Cai et al., Highly sensitive non-stripping gold nanoparticles-based chemiluminescent detection of DNA hybridization coupled to magnetic beads, Analyst 135 (3) 615-620 (2010).
Cui et al., Synthesis, Characterization, and Electrochemiluminescence of Luminol-Reduced Gold Nanoparticles and Their Application in a Hydrogen Peroxide Sensor, Chem Eur J 13 (24) 6975-6984 (2007).
Duan et al., Size-Dependent Inhibition and Enhancement by Gold Nanoparticles of Luminol-Ferricyanide Chemiluminescence, J Phys Chem C 111 (12) 4561-4566 (2007).
Hostetler et al., Alkanethiolate Gold Cluster Molecules with Core Diameters from 1.5 to 5.2 nm: Core and Monolayer Properties as a Function of Core Size, Langmuir 14 (1) 17-30 (1998).
Hostetler et al., Monolayers in three dimensions: synthesis and electrochemistry of omega-functionalized alkanethiolate-stabilized gold cluster compounds, J Am Chem Soc 118 (17) 4212-4213 (1996).
Jain et al., Calculated absorption and scattering properties of gold nanoparticles of different size, shape, and composition: applications in biological imaging and biomedicine, J Phys Chem B 110 (14) 7238-7248 (2006).
Kim et al., Synthesis and Thermally Reversible Assembly of DNA-Gold Nanoparticle Cluster Conjugates, Nano Lett 9 (12) 4564-4569 (2009).
Kim, H.-S.; Pyun, J.-C., Procedia Chemistry 1 (1) 1043-1046 (2009).
Knox Van Dyke et al., Luminescence Biotechnology: Instrumentation and Applications, CRC Press, New York (2001).
Lee et al., A DNA-Gold Nanoparticle-Based Colorimetric Competition Assay for the Detection of Cysteine, Nano Lett 8 (2) 529-533 (2008).
Li et al., Colorimetric detection of DNA sequences based on electrostatic interactions with unmodified gold nanoparticles, Proc Natl Acad Sci 101 (39) 14036-14039 (2004).
Li et al., Immobilization of glucose oxidase onto gold nanoparticles with enhanced thermostability, Biochem Biophys Res Commun 355 (2) 488-493 (2007).
Li et al., Label-free colorimetric detection of specific sequences in genomic DNA amplified by the polymerase chain reaction, J Am Chem Soc 126 (35) 10958-10961 (2004).
Liu et al., Chemiluminescence detection for a microchip capillary electrophoresis system fabricated in poly(dimethylsiloxane), Anal Chem 75 (1) 36-41 (2003).
Mao et al., Disposable nucleic acid biosensors based on gold nanoparticle probes and lateral flow strip, Anal Chem 81 (4) 1660-1668 (2009).
Mirkin et al., A DNA-based method for rationally assembling nanoparticles into macroscopic materials, Nature 382 (6592) 607-609 (1996).
Prilutsky et al., Differentiation between Viral and Bacterial Acute Infections Using Chemiluminescent Signatures of Circulating Phagocytes, Anal Chem 83 (11) 4258-4265 (2011).
Rechberger et al., Optical properties of two interacting gold nanoparticles, Opt Commun 220 (1-3) 137-141 (2003).
Ronaghi et al., A sequencing method based on real-time pyrophosphate, Science 281 (5375) 363-365 (1998).
Schmid et al., Gold nanoparticles: assembly and electrical properties in 1-3 dimensions, Chem Commun (6) 697-710 (2005).

Silverstein et al., Spectrometric Identification of Organic Compounds, 4th ed. John Wiley and Sons, New York (1981).
Su et al., Control of metal nanoparticles aggregation and dispersion using PNA and PNA-DNA complexes, and its use for colorimetric DNA detection, ACS Nano 3 (9) 2751-2759 (2009).
Su et al., Recent advances in chemiluminescence, Appl Spectrosc Rev 42 (2), 139-176 (2007).
Taton et al., Scanometric DNA Array Detection with Nanoparticle Probes, Science 289 (5485) 1757-1760 (2000).
Tsukagoshi et al., Direct detection of biomolecules in a capillary electrophoresis-chemiluminescence detection system, Anal Chem 76 (15) 4410-4415 (2004).
White et al., Chemiluminescence of Luminol and Related Hydrazides: The Light Emission Step, J Am Chem Soc 86 (5) 941-942 (1964).
White et al., Chemilunimescence of Luminol: The Chemical Reaction, J Am Chem Soc 86 (5) 940-941 (1964).
White, E. H.; Roswell, D. F., Acc Chem Res, 3 (2), 54-62 (1970).
Xia et al., Colorimetric detection of DNA, small molecules, proteins, and ions using unmodified gold nanoparticles and conjugated polyelectrolytes, Proc Natl Acad Sci 107 (24) 10837-10841 (2010).
Yang, X.; Guo, Y.; Wang, A., Anal Chim Acta 666 (1-2) 91-96 (2010).
Yuan et al., Determination of sub-nanomolar levels of hydrogen peroxide in seawater by reagent-injection chemiluminescence detection, Anal Chem 71 (10) 1975-1980 (1999).
Zhang et al., Gold nanoparticle-catalyzed luminol chemiluminescence and its analytical applications, Anal Chem 77 (10) 3324-3329 (2005).
Zhao et al., Chemiluminescence immunoassay, TrAC Trends Anal Chem 28 (4) 404-415 (2009).

We claim:

1. A kit for detecting an analyte in a sample comprising:
a light-shielding container having a fiberoptic cable for transmitting light generated within said light-shielding container to a photodetector;
a plurality of functionalized nanoparticles deposited in solid form on or within a support, said support located within said light-shielding container;
wherein said functionalized nanoparticles comprise nanoparticles covalently attached with one or more chemiluminescent moieties; and
a reagent system which causes said chemiluminescent moieties to produce light in the presence of said reagent system and said analyte in said sample.

2. The kit of claim 1 wherein said functionalized nanoparticle comprises a gold nanoparticle having a diameter between about 2 and 50 nm.

3. The kit of claim 1 wherein said functionalized nanoparticle comprises a gold nanoparticle having a diameter between about 5 and 15 nm.

4. The kit of claim 1 wherein said nanoparticle is functionalized with luminol.

5. The kit of claim 1 wherein said luminol is attached to said nanoparticle via a linker having between 8 and 20 carbons.

6. The kit of claim 1 wherein said support is a multiwell plate having a plurality of wells, wherein said wells have a plurality of nanoparticles deposited on a surface of said well, wherein said analyte is blood, and wherein said reagent system comprise an oxidant and a base.

7. The kit of claim 6 wherein said reagent system comprises hydrogen peroxide and sodium hydroxide.

8. The kit of claim 1 wherein said functionalized nanoparticles are further functionalized with a first oligonucleotide probe.

9. The kit of claim 8 wherein said first oligonucleotide probe is capable of selectively hybridizing to a nucleic acid of an RNA virus.

10. The kit of claim 9 wherein said RNA virus is a Hepatitis C virus.

11. The kit of claim 10 wherein said first oligonucleotide probe is capable of selectively hybridizing to the X-tail of the Hepatitis C virus.

12. The kit of claim 8 wherein said first oligonucleotide probe is capable of selectively hybridizing to a nucleic acid of a bacterium.

13. The kit of claim 12 wherein said bacterium is Chlamydia trachomatis.

14. The kit of claim 8 wherein said first oligonucleotide probe is selected from the group consisting of SEQ. ID NO: 1-16.

15. The kit of claim 8 wherein said first oligonucleotide probe is attached to said nanoparticle via a linker having between 8 and 20 carbons.

16. The kit of claim 8 wherein said support is a test strip, wherein said functionalized nanoparticles are deposited in solid form on an application pad portion of said test strip, wherein said analyte is a virus nucleic acid, and wherein said reagent system comprise an oxidant, a base, and a metal ion catalyst.

17. The kit of claim 16 wherein said test strip further comprises a test pad region having a second oligonucleotide probe capable of selectively hybridizing said virus nucleic acids.

18. The kit of claim 17 wherein said test strip further comprises a control pad region having a third oligonucleotide probe capable of selectively hybridizing said first oligonucleotide probe.

19. A kit for detecting a bacterium or virus in a sample comprising:
a light-shielding container having a fiberoptic cable for transmitting light generated within said light-shielding container to a photodetector;
a support located within said light-shielding container, said support having a sample application region, a test region, and a control region;
a plurality of first functionalized nanoparticles deposited in solid form on or within said sample application region of said support, wherein said first functionalized nanoparticles comprise nanoparticles covalently attached to a chemiluminescent moiety and a first oligonucleotide probe capable of selectively hybridizing to bacterium or virus nucleic acids;
a plurality of second particles functionalized with a second oligonucleotide probe capable of selectively hybridizing to said bacterium or virus nucleic acids, said second particles immobilized on or within said test region of said support;
a plurality of third particles functionalized with a third oligonucleotide probe capable of selectively hybridizing to said first oligonucleotide probe, said third particles immobilized on or within said control region of said support; and
a reagent system which causes said chemiluminescent moiety to produce light in the presence of said reagent system and said first functionalized nanoparticles.

20. The kit of claim 19 wherein said first oligonucleotide probe is capable of selectively hybridizing to an RNA virus.

21. The kit of claim 20 wherein said RNA virus is a Hepatitis C virus.

22. The kit of claim 21 wherein said first oligonucleotide probe is capable of selectively hybridizing to the X-tail of the Hepatitis C virus.

23. The kit of claim 21 wherein said first oligonucleotide probe and said second oligonucleotide probe are both capable of selectively hybridizing to the X-tail of the Hepatitis C virus.

24. The kit of claim 19 wherein said first oligonucleotide probe is attached to said nanoparticle via a linker having between 8 and 20 carbons.

25. The kit of claim 19 wherein said first oligonucleotide probe is capable of selectively hybridizing to a nucleic acid of a bacterium.

26. The kit of claim 25 wherein said bacterium is Chlamydia trachomatis.

27. The kit of claim 19 wherein said first oligonucleotide probe is selected from the group consisting of SEQ. ID NO: 1-16.

28. A method for detecting blood in a sample comprising:
providing support having a plurality of functionalized nanoparticles deposited on or within support in solid form, wherein said functionalized nanoparticles comprise nanoparticles covalently attached to a chemiluminescent moiety;
contacting said sample with said functionalized nanoparticles in the presence of a reagent system having an oxidant and a base;
determining whether light is generated when said functionalized nanoparticles are contacted with said sample in the presence of said reagent system;
wherein generated light is an indication that the sample contains blood.

29. The method of claim 28 wherein said functionalized nanoparticle comprises a gold nanoparticle having a diameter between about 2 and 50 nm.

30. The method of claim 28 wherein said functionalized nanoparticle comprises a gold nanoparticle having a diameter between about 5 and 15 nm.

31. The method of claim 28 wherein said nanoparticles are functionalized with luminol.

32. The method of claim 28 wherein said luminol is attached to said nanoparticles via a linker having between 8 and 20 carbons.

33. The method of claim 28 wherein said support is a multiwell plate having a plurality of wells, wherein said wells have a plurality of said functionalized nanoparticles deposited on a surface of said well, and wherein said reagent system comprises hydrogen peroxide and sodium hydroxide.

34. A method for detecting a target bacterium or virus in a sample comprising:
providing support having a plurality of first functionalized nanoparticles deposited on or within support in solid form, wherein said functionalized nanoparticles comprise nanoparticles covalently attached to a chemiluminescent moiety and a first oligonucleotide probe capable of selectively hybridizing to target bacterium or virus nucleic acids;
flowing said sample along said support such that the first oligonucleotide probe of the functionalized nanoparticle selectively hybridizes to said target bacterium or virus nucleic acid to form a hybridized functionalized nanoparticle if the target bacterium or virus nucleic acid is present in said sample;
contacting said sample in the presence of a reagent system having an oxidant, a base, and a metal catalyst;
determining whether light is generated when said sample is contacted with a reagent system;
wherein generated light is an indication that the sample contains the target bacterium or virus nucleic acid.

35. The method of claim 34 wherein said first oligonucleotide probe is capable of selectively hybridizing to the nucleic acid of an RNA virus.

36. The method of claim 34 wherein said virus is a Hepatitis C virus.

37. The method of claim 34 wherein said first oligonucleotide probe is capable of selectively hybridizing to the X-tail of the Hepatitis C virus.

38. The method of claim 34 wherein said first oligonucleotide probe is capable of selectively hybridizing to a nucleic acid of a bacterium.

39. The method of claim 38 wherein said bacterium is Chlamydia trachomatis.

40. The method of claim 34 wherein said first oligonucleotide probe is selected from the group consisting of SEQ. ID NO: 1-16.

41. The method of claim 34 wherein said first oligonucleotide probe is attached to said nanoparticle via a linker having between 8 and 20 carbons.

42. The method of claim 34 wherein said support is a test strip, wherein said functionalized nanoparticles are deposited on along an application region of said test strip, wherein said flowing step comprises applying said sample to said application region and permitting said sample to flow by capillary action along said support.

43. The method of claim 42 wherein said test strip further comprises a test region having a second oligonucleotide probe capable of selectively hybridizing said target bacterium or virus nucleic acids immobilized on said test region, and where said test strip further comprises a control region having a third oligonucleotide probe capable of selectively hybridizing first oligonucleotide probe immobilized in said control region; and comprising the steps of flowing said sample containing said hybridized functionalized nanoparticles across said test region to capture said hybridized functionalized nanoparticles and flowing said sample across said control region to capture excess first functionalized nanoparticles which are not hybridized.


SEQUENCE LISTING (16 sequences)

SEQ ID NO: 1 - DNA, 22 bp, Hepatitis C virus
gtggccccatcttagccctagt

SEQ ID NO: 2 - DNA, 25 bp, Hepatitis C virus
acggctagctgtgaaaggtccgtga

SEQ ID NO: 3 - DNA, 35 bp, Influenza Hemagglutinin virus
gtctccctgggggcaatcagtttctggatgtgctc

SEQ ID NO: 4 - DNA, 34 bp, Influenza Hemagglutinin virus
caaatgcagacacattatgtataggttatcatgc

SEQ ID NO: 5 - DNA, 41 bp, Influenza Hemagglutinin virus
acagtactagaaaagaatgtaacagtaacacactctgttaa

SEQ ID NO: 6 - DNA, 29 bp, Influenza Hemagglutinin virus
agagagcaattgagctcagtgtcatcatt

SEQ ID NO: 7 - DNA, 30 bp, Influenza Neuraminidase virus
cactatgaggaatgctcctgttatcctgat

SEQ ID NO: 8 - DNA, 29 bp, Influenza Neuraminidase virus
tctaatggagcaaatggagtaaaaggatt

SEQ ID NO: 9 - DNA, 32 bp, Influenza Neuraminidase virus
ggcaatggtgtttggatagggagaactaaaag

SEQ ID NO: 10 - DNA, 64 bp, Chlamydia trachomatis
tcgcatgctcaatagtgcgacttgtgctgctggcggcataggattgttaacaccagtggtatgc

SEQ ID NO: 11 - DNA, 31 bp, Chlamydia trachomatis
atgaacacactcagttttagaaacgcttttg

SEQ ID NO: 12 - DNA, 28 bp, Chlamydia trachomatis
ggcagttgctgtggccactatattggcc

SEQ ID NO: 13 - DNA, 28 bp, Chlamydia trachomatis
tagcggcatctttattcttcggggtagg

SEQ ID NO: 14 - DNA, 30 bp, Chlamydia trachomatis
ttggaggagtgctgactacagaagctgtga

SEQ ID NO: 15 - DNA, 44 bp, Chlamydia trachomatis
catcgatcacaaactttgatgtggaacaacttatgctgtaaaac

SEQ ID NO: 16 - DNA, 57 bp, Chlamydia trachomatis
gcagaggttgagcagaaaatctcgacagctagtgcaaatgccaaaagcaatgataag